(19) United States
(12) Patent Application Publication
(10) Pub. No.: US 2017/0136102 A1
(43) Pub. Date: May 18, 2017

(54) PHAGE-DERIVED COMPOSITIONS FOR IMPROVED MYCOBACTERIAL THERAPY

(71) Applicants: Umender Kumar SHARMA, Malleshwaram, Bangalore (IN); GangaGen, Inc., Newark, CA (US)

(72) Inventor: Umender Kumar SHARMA, Malleshwaram, Bangalore (IN)

(21) Appl. No.: 15/127,521

(22) PCT Filed: Mar. 26, 2015
(86) PCT No.: PCT/IN2015/000144

§ 371 Date: Sep. 20, 2016

(30) Foreign Application Priority Data
Mar. 27, 2014 (IN) 1647/CHE/2014

(51) Int. Cl. A61K 38/47; A61K 31/4409; A61K 31/133; A61K 31/496

(52) U.S. Cl. CPC A61K 38/47; A61K 31/496; A61K 31/4409; A61K 31/133; C12Y 302/01

(57) ABSTRACT

Methods and compositions for the treatment of mycobacteria infections, particularly antibiotic resistant strains. Of particular use in treating tuberculosis infections, including dormant or difficult to treat forms.

### FIELD OF THE INVENTION

The present disclosure relates to the field of biotechnology, particularly regarding therapy and treatment of mycobacteria infections. Compositions useful for treatment of various mycobacteria infections are described.

### BACKGROUND OF THE INVENTION

Tuberculosis (TB) caused my M. tuberculosis is an important human disease which is responsible for significant morbidity and mortality all over the world. According to recent estimates there are more than 9 million TB cases detected worldwide every year (World Health Organization, Global tuberculosis report 2012, World Health Organization Document 2012; WHO/HTM/TB/2012.6:1-272). With the spread of HIV, the incidence of TB cases has increased (De Cock, et al. (1992) “Tuberculosis and HIV infection in sub-Saharan Africa” JAMA268:1581-1587; de Jong, et al. (2004)“Clinical management of tuberculosis in the context of HIV. Infection” Ann. Rev. Med. 55:283-301. In majority of the cases M. tuberculosis causes lung disease, however, cases of extra pulmonary tuberculosis are also quite common. Unlike other bacterial infections, treatment of TB is very cumbersome and it requires administration of at least four drugs for a period of six months (Global Alliance for TB Drug Development (2008)“Handbook of Anti-Tuberculosis Agents” Tuberculosis 88:85-170). Because of complex treatment regimen, a number of patients do not complete the required treatment course of six months. In addition, there is tendency towards not taking all the prescribed drugs regularly. Because of these issues, bacteria get exposed to sub-optimal doses of various drugs resulting in emergence of drug resistant bacteria. Bacterial strains resistant to two frontline drugs (MDR) or to many frontline and second line drugs (XDR) are spreading throughout the world (Mondal (2013) Genetics & Transmission Factors of Drug Resistance in Mycobacteria Lambert Academic Publishing ISBN-10: 365948752X, ISBN-13: 978-3659487521). Treatment of MDR and XDR TB is even more complicating as it involves administration of several drugs for up to 18 months. Thus there is an urgent need to discover new TB drugs (Walter, et al. (2012) “Translating basic science insight into public health action for multidrug- and extensively drug-resistant tuberculosis” Respirology 17:772-791).

The long duration of TB therapy has been attributed to the presence of non-replicating or slowly replicating bacteria which are phenotypically resistant to the action of anti-TB drugs(Lienhardt, et al. (2012) “New drugs for the Treatment of Tuberculosis: Needs, Challenges, Promise, and Prospects for the Future” J. Infectious Diseases 2012:205 (Suppl. 2) S241-49; and Sacchettini, et al. (2008) “Drugs versus bugs: in pursuit of the persistent predator Mycobacterium tuberculosis” Nat. Revs. Microbiology 6:41-52). All humans exposed to M. tuberculosis do not end up getting the disease. A large majority of them carry M. tuberculosis in the body in a sub-clinical form called latent infection. Most people carrying latent TB may not get the disease, only a fraction of them will get an active disease. A number of factors such as malnutrition, stress, diabetes, etc. are the predisposing factors for development of disease. Since duration of treatment is very long, which is due to the presence of persistors, a lot of effort has gone into understanding the mechanism of persistence. One important hallmark of latency/dormancy has been thought to be the presence of non-replicating or slowly replicating bacteria. Hence drugs which show inhibitory properties on non-replicating M. tuberculosis cells are considered important for reducing the duration of TB therapy. A number of in-vitro and ex-vivo models have been proposed for mimicking the latent or dormant state of bacteria. These include microaerophilic or anaerobic models, nutrient starvation models or models using Streptomycin dependent strains of M. tuberculosis. (Wayne (1994) “Dormancy of M. Tuberculosis and latency of disease” Eur. J. Clin. Microbiol. Infect. Dis. 13:908-14; Kapoor, et al. (2013) “Human Granuloma In Vitro Model, for TB Dormancy and Resuscitation” PLOSone 8:e53657.

Other diseases caused by mycobacteria include leprosy caused by M. leprae and diseases caused by non-tuberculous mycobacteria (NTM). NTM can cause pulmonary and non-pulmonary disease including skin ulcers, soft tissue and lymphatic infections (Grange (2007)“Environmental mycobacteria” in Greenwood, et al. (Eds.) Medical Microbiology (17th ed.), pp. 221-227. Buruli ulcer caused by M. ulcerans is a difficult to treat infection prevalent in Africa and some other parts of the world. See Kazda, et al. (2009) The Ecology of Mycobacteria: Impact on Animal's and Human's Health Springer ISBN-10: 1402094124, ISBN-13: 978-1402094125; Simpson (2005) The NTM Handbook: A Guide for Patients with Nontuberculous Mycobacterial Infections including MAC Ginkgo Publishing ISBN-10: 0615365612, ISBN-13: 978-0615365619; Converse, et al. (2011) “Treating Mycobacterium ulcerans disease (Buruli ulcer): from surgery to antibiotics, is the pill mightier than the knife?” Future Microbiol. 6:1185-1198; Trigo, et al. (2013) “Phage Therapy Is Effective against Infection by Mycobacterium ulcerans in a Murine Footpad Model” PLoS Negl. Trop. Dis. 7:e2183.

### BRIEF SUMMARY OF THE INVENTION

Provided herein are methods, combination therapies, and compositions for improved eradication of mycobacteria. For example, provided are methods for treating a subject having a mycobacterial infection, comprising administering to the subject: a) an outer membrane acting biologic; and b) a mycobacterial chemotherapeutic, thereby treating the subject. In some embodiments, the outer membrane acting biologic and mycobacterial therapeutic act synergistically to reduce mycobacteria. In some embodiments, the method does not include administering a fusion protein comprising a peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP. In some embodiments, the outer membrane acting biologic is not derived from phage LysA (e.g., isolated LysA, tagged, mutated, or truncated LysA, or recombinantly produced LysA). In some embodiments, the outer membrane acting biologic is derived from phage LysB (e.g., isolated LysB, tagged, mutated, or truncated LysB, or recombinantly produced LysB retaining outer membrane degrading activity).

In some embodiments, less than 90% of the standard dose of mycobacterial chemotherapeutic is administered (e.g., less than 80%, 70%, 50%, 25%, or 10%) while retaining the anti-mycobacterial activity of 100% of the standard dose in the absence of the outer membrane acting biologic. In some embodiments, the mycobacterial chemotherapeutic is administered for less than 90% of the standard duration of treatment (e.g., less than 80%, 70%, 50%, 25%, or 10%) while retaining the anti-mycobacterial activity of 100% of the standard duration in the absence of the outer membrane acting biologic. In some embodiments, the FIC index is less than 0.6 (e.g., less than 0.5, 0.3, 0.1, 0.07, 0.05, 0.03, or 0.01).

In some embodiments, the outer membrane acting biologic and mycobacterial chemotherapeutic are both administered within a period of about 2 days. In some embodiments, the outer membrane acting biologic and mycobacterial chemotherapeutic are both administered within a period of about 28, 21, 14, 7, 5 days, or less than about 1 day (e.g., within about 12, 6, 2, or 1 hour). In some embodiments the outer membrane acting biologic is administered before the mycobacterial chemotherapeutic or both are administered simultaneously. In some embodiments, the outer membrane acting biologic, mycobacterial chemotherapeutic, or both are administered in a slow release formulation.

In some embodiments, the method further comprises administering a) a biologic or therapeutic compound which increases accessibility of the mycobacterial chemotherapeutic to a mycobacterial cell inside a macrophage or monocyte; b) a biologic or therapeutic compound which increases accessibility of the mycobacterial chemotherapeutic to the interior of a granuloma; or c) a biologic or therapeutic compound that increases permeability of a macrophage or monocyte. In some embodiments, the method further comprises administering a therapeutic compound that increases the permeability of a macrophage, monocyte, or granuloma.

In some embodiments, the subject immunosuppressed, e.g., HIV positive, or has cystic fibrosis, alpha-1 antitrypsin deficiency, Marfan's syndrome, or Primary Ciliary Dyskenesia. In some embodiments, the subject is a mammal (e.g., human, non-human primate, etc.), reptile, amphibian, or fish.

In some embodiments, the mycobacterial infection is a tuberculosis infection; environmental mycobacteria; atypical mycobacteria; a mycobacteria other than tuberculosis (MOTT) or non-tuberculosis mycobacteria (NTMB); latent; active; disseminated; extrapulmonary or lymphatic; multidrug resistant; a post-traumatic abscess, swimming pool granuloma, Buruli ulcer, or leprosy. In some embodiments, the infection is detected by chest X-ray; microbiological culture (e.g., of a biological sample from the subject); evaluation of sputum or biopsy; histology; PCR or other nucleic acid hybridization assay; a Purified Protein Derivative test; a Mantoux test; symptoms of mycobacterial infection; or immunoassay (e.g., ELISA).

In some embodiments, the outer membrane acting biologic: decreases the amount of mycobacterial chemotherapeutic or duration of mycobacterial chemotherapeutic treatment required to treat the mycobacterial infection in the subject; increases permeability of a granuloma; acts on a mycolic acid or lipoarabinomanin; acts on a mycobacterial cell wall or outer membrane; is an esterase; is a lipase; is a cutinase; is an alpha/beta hydrolase (e.g., Cousin, et al. (1997) “The α/β fold family of proteins database and the cholinesterase gene server ESTHER” Nucleic Acids Research 25:143-146); is mycolylarabinogalactan esterase; is phage LysB; is D29 phage LysB; or is in a sterile or buffered formulation.

In some embodiments, the mycobacterial chemotherapeutic is one or more of a front line mycobacterial therapeutic such as isoniazid, pyrazinamide, ethambutol, or rifampin; a second line mycobacterial therapeutic such as a fluoroquinolone (e.g., ciprofloxacin, levefloxacin, moxifloxacin), a cyclic peptide (e.g., capromycin, viomycin, enviomycin), a thioamide (e.g., ethionamide, prothionamide), cycloserine, terizidone, an aminoglycoside, PAS, kanamycin, capreomycin, amikacin, or streptomycin; a third or subsequent line mycobacterial therapeutic; a microlide, a β-lactam, a β-lactamase inhibitor, clavulenic acid, trimethoprim, sulfamethoxazole; clarithromycin, rifampicin, rifabutin, amikacin, azithromycin, moxifloxacin; and diarylquinoline, dedaquiline, TMC207, a nitroimdazole, PA-824, OPC-67683, an oxazolidinone, linezolid, sutezolid, AZD5847, BTZ043, and SQ109.

In some embodiments, the method reduces mycobacterial levels in the subject with a lower dose of mycobacterial chemotherapeutic than would be required in the absence of the outer membrane acting biologic; reduces mycobacterial levels in the subject with a shorter duration of treatment with the mycobacterial chemotherapeutic than would be required in the absence of the outer membrane acting biologic; reduces side effects compared to administration of the mycobacterial chemotherapeutic alone; reduces mycobacterial levels in the subject with a reduced number of mycobacterial chemotherapeutics than would be required in the absence of the outer membrane acting biologic.

Further provided are kits, e.g., comprising compartments or therapeutic composition(s), comprising an outer membrane acting biologic and a mycobacterial chemotherapeutic as described above. In some embodiments, the outer membrane acting biologic and a mycobacterial chemotherapeutic are in separate compartments (e.g., blister packs). In some embodiments, the outer membrane acting biologic and a mycobacterial chemotherapeutic are combined in a single compartment or therapeutic composition. In some embodiments, the kit comprises more than one mycobacterial chemotherapeutic, e.g., combined with the outer membrane acting biologic or separate. In some embodiments, the kit further comprises a therapeutic compound that increases the permeability of a macrophage, monocyte, or granuloma. In some embodiments, the kit includes the outer membrane acting biologic and mycobacterial chemotherapeutic(s) packaged in single dosage forms, either together or separately.

Further provided are methods of treating a subject having a mycobacterial infection, comprising administering to the subject a LysB biologic, wherein the LysB biologic reduces mycobacteria, thereby treating the subject. In some embodiments, the method further comprises administering a mycobacterial chemotherapeutic, e.g., either in succession or simultaneously, e.g., in a single therapeutic composition.

As indicated above, the mycobacterial infection can be tuberculosis. In some embodiments, the mycobacterial infection can be latent; active; disseminated; extrapulmonary or lymphatic; multidrug resistant; a post-traumatic abscess, swimming pool granuloma, Buruli ulcer, or leprosy. In some embodiments, the infection is detected by chest X-ray; microbiological culture (e.g., of a biological sample from the subject); evaluation of sputum or biopsy; histology; PCR or other nucleic acid hybridization assay; a Purified Protein Derivative test; a Mantoux test; symptoms of mycobacterial infection; or immunoassay (e.g., ELISA). In some embodiments, the subject immunosuppressed, e.g., HIV positive. In some embodiments, the subject is a mammal (e.g., human, non-human primate, etc.), reptile, amphibian, or fish.

### DETAILED DESCRIPTION OF THE INVENTION

### I. Introduction

Mycobacteria are aerobic and non-motile bacteria (except for the species Mycobacterium marinum, which has been shown to be motile within macrophages) that are characteristically acid-alcohol-fast. Mycobacteria generally do not contain endospores or capsules and are usually considered Gram-positive. While mycobacteria do not seem to fit the Gram-positive category from an empirical standpoint (i.e., in general, they do not retain the crystal violet stain well), they are classified as an acid-fast Gram-positive bacterium due to their lack of an outer cell membrane.

Human disease-associated bacteria are often divided into four groups (Runyon classification; see Grange (2007) “Environmental mycobacteria” pp. 221-227 in Greenwood, et al. (Eds.) Medical Microbiology (17th ed.) Elsevier, ISBN 9780443102097) which are the Photochromogens, which develop pigments in or after being exposed to light, and include the examples M. kansasii, M. simiae, and M. marinum; the Scotochromogens, which become pigmented in darkness, and include the examples M. scrofulaceum and M. szulgai; the Non-chromogens, which include a group of prevalent opportunistic pathogens called M. avium complex (MAC), and other examples of M. ulcerans, M. xenopi, M. malmoense, M. terrae, M. haemophilum, and M. genavense; and Rapid growers, which include four well recognized pathogenic rapidly growing non-chromogenic species: M. chelonae, M. abscessus, M. fortuitum, and M. peregrinum; and other rare examples of M. smegmatis and M. flavescens.

The structure of the mycobacteria cell wall is complex, and is not easily permeable to small molecule antibiotics. See, e.g., Sarathy, et al. (2012) “The Role of Transport Mechanisms in Mycobacterium Tuberculosis Drug Resistance and Tolerance” Pharmaceuticals 5:1210-1235, doi: 10.3390/ph5111210; and Gangadharam (2012) Mycobacteria. I Basic Aspects Springer ASIN: BOOEZ16T9G. In particular, the cell wall serves as a permeability barrier to accessibility to common mycobacteria chemotherapeutics. See, e.g., Daffe and Reyrat (eds. 2008) The Mycobacterial Cell Envelope ASM Press ISBN-10: 1555814689, ISBN-13: 9781555814687. The relative insensitivity of target mycobacteria infections to treatment result in having to increase doses of chemotherapeutics, and often to combine many different chemotherapeutics because subjects are often intolerant to the side effects of high dose treatment. Compliance with therapeutic directives is often low, which requires careful monitoring to ensure the drug combinations are actually administered. If orally administered, the subjects may often need to be monitored to ensure the stomach contents are not emptied by vomiting or the like. In particular, a great difficulty in treatment of a subject is the careful tracking and monitoring to ensure the subject actually complies with treatment, especially at times when the symptoms of treatment appear worse than the short term effects of diminishing disease. With more effective treatment, the time and effort to track subjects may be decreased, the dose of chemotherapeutics may be decreased, and/or the number of different drugs to be administered (each causing negative side effects) might be lessened. A great need still exists for more effective mycobacteria infection treatment strategies as multidrug and extensively drug resistant isolates are becoming more common. See Lienhardt, et al. (2012) “New Drugs for the Treatment of Tuberculosis: Needs, Challenges, Promise, and Prospects for the Future” J. Infect. Dis. 205(Suppl. 2):S241; Sacchettini, et al (2008) “Drugs versus buts: in pursuit of the persistent predator Mycobacterium tuberculosis” Nature Rev. Microbiol. 6:41-52; and Walter, et al. (2012) “Translating basic science insight into public health action for multidrug- and extensively drug-resistant tuberculosis” Respirology 17:772-791, doi: 10.1111/j.1440-1843.2012.02176.

### II. Anti-Mycobacterial Treatments and Therapies

As described above, the present disclosure is based, in part, upon the recognition that the combination of a biologic with mycobacteria chemotherapeutics has synergistic effects on the targets. Described herein are a number of biologics which act synergistically with at least one or several standard mycobacteria chemotherapeutics, and which can be used to decrease the dose, duration, or number of different chemotherapeutics used for treatment.

Many mycobacteria infections remain dormant or minimally active, largely because they may be contained within the lysosomes of macrophages which have engulfed the target mycobacteria. The mycobacteria can avoid being killed by remaining within the macrophages. These mycobacteria also may be sequestered from the drug treatment by the macrophage cells. Means to penetrate such barriers are also provided herein.

Mycobacteria-engulfed macrophages can cluster into nodules known as granulomas. These are additional structures that the host organism can use to wall off the macrophages and mycobacteria to prevent their escape to other parts of the body. The granuloma often is walled away from the body by a fibroblast or other cell layers, which often also become calcified. These granulomas also serve as permeability barriers to prevent accessibility of therapeutics to the target mycobacteria, which may also be in latent or near dormant physiological states. The present disclosure addresses various means to increase local concentration of mycobacteria chemotherapeutics at the cell membrane of target cells.

### III. Definitions

Two or more therapeutic entities exhibit “synergy” when the combinations exhibit a greater effect than the additive effects of the individual entities, e.g., a substantially better effect than would be expected based on the entities' individual activities. For example, drug synergy occurs when two or more drugs can interact in ways that enhance or magnify one or more positive or advantageous effects of those drugs compared to use when not combined together. This is sometimes exploited in combination preparations, where the therapeutics are admixed or combined into a single formulation, which results in administering them together. Alternatively, the individual compositions may be administered separately, e.g., where each is substantially pure, so they are present in the body at the same time. Negative effects of combination are a form of contraindication, e.g., adverse effects from the combinations.

Measures of synergy typically measure the amount of effect of each component alone when compared to a combination. See, e.g., Geary (2013) “Understanding synergy” Am. J Physiol. Endocrin. Metab. 304:E237-E253, DOI: 10.1152/ajpendo.00308.2012; Torella, et al. (2010) “Optimal Drug Synergy in Antimicrobial Treatments” PLoS Comput. Biol. 6:e1000796, PMCID: PMC2880566; and Tallarida (2001) “Drug Synergism: Its Detection and Applications” J. Pharmacology and Expt'l Therapeutics 298:865-872. Standard measures of synergy include the Fractional Inhibitory Concentration (FIC) index. See Konate, et al. (2012) “Antibacterial activity against β-lactamase producing Methicillin and Ampicillin-resistants Staphylococcus aureus: fractional Inhibitory Concentration Index (FICI) determination” Annals of Clinical Microbiology and Antimicrobials 11:18. FIC can be calculated from the Minimal Inhibitory Concentrations (MIC) of two drugs, X and Y, and of their combinations, as described below. The FIC index is a measure of synergy, with an index value of 0.5 typically a threshold number. In other embodiments, the threshold for “synergy” designation may be 0.4, 0.3, 0.2, 0.1, etc., over a statistically useful determination.

Drug synergy can occur both in biological activity and because of pharmacokinetics, e.g., where one entity significantly affects the pharmacokinetic properties of the other. Shared metabolic enzymes can cause drugs to remain in the bloodstream much longer in higher concentrations than if individually taken, e.g., where both entities compete for a deactivating mechanism. The biological activity synergy is that normally observed in these combinations.

Permeability or accessibility of target mycobacteria cells to therapeutic agents may be achieved by affecting the outer cell wall of the mycobacteria. Mycobacterium species share a characteristic cell wall, thicker than in many other bacteria, which is hydrophobic, waxy, and rich in mycolic acids/mycolates and arabinogalactan, etc. These are often referred to as a “mycobacterial outer membrane”. Inside this mycobacteria outer membrane is a peptidoglycan layer. The cell wall makes a substantial contribution to the hardiness of this genus. Inside the peptidoglycan layer is the cell membrane. See, e.g., Daffe and Reyrat (eds. 2008) The Mycobacterial Cell Envelope ASM Press ISBN-10: 1555814689, ISBN-13: 978-1555814687; Lopez-Marin (2012) “Nonprotein Structures from Mycobacteria: Emerging Actors for Tuberculosis Control” Clinical and Developmental Immunology 2012:ID 917860; Favrot and Ronning (2012) “Targeting the mycobacterial envelope for tuberculosis drug development” Expert Rev. AntiInfect. Ther. 10:1023-1036; and Jackson, et al. (1999)“Inactivation of the antigen 85C gene profoundly affects the mycolate content and alters the permeability of the Mycobacterium tuberculosis cell envelope” Molecular Microbiology 31:1573-1587.

“Coordinated” therapy exists when two or more therapies are used together. The coordinated therapy may be simultaneously applied, or sequentially. Where the pharmacological effect of one remains when the other is provided, they will work together during the period when both are present. In certain embodiments, the different therapies may be administered in succession, which may be specifically ordered or randomly ordered. In some cases, a therapy might incorporate other than a drug, e.g., which might be a procedure such as massage or special breathing methods.

For a therapeutic drug, “administering” is dosing to the subject, and may include many means of administration. Administration can be oral, topical, local, systemic, parenteral, non-parenteral, etc. In many cases, the administering will involve inserting drug into the person, e.g., by injection, inhalation, topical absorption, or other.

Two or more drugs may be provided by “simultaneous” administration, e.g., where both are administered with a short period. The administration of drugs might be coadministered in a single formulation, or each administered in rapid succession. Where administration may involve some period of time, they may be successively administered within one medical procedure or visit. Typically a visit may take up to an hour, or the administration procedure may be an infusion, which may extend for a few hours. In other embodiments, the administrations may be virtually instantaneous, e.g., swallowing of a pill or injection of a small volume.

In some embodiments, the drugs might be provided by “successive” administration, e.g., within reasonably short periods, e.g., hours, or within 2, 3, 5, 7, 10, 14, 17, 21, 24, 18, 30, 34, 38 days, etc. In some embodiments, the drugs are administered close enough in time to retain synergistic effect. In some cases, the drugs may be administered in either order, while in others, one will be indicated to be administered before another. Because the pharmacokinetics of different drugs may differ, the combination may have special temporal windows where both are present at the correct site in appropriate concentrations.

In certain embodiments, the presently disclosed compositions and methods incorporate an additional means to achieve a function of increasing the permeability of a mycobacteria granuloma. A granuloma is an inflammation found in many diseases. It is a collection of immune cells, typically macrophages or monocytes, but may include other cell types. Granulomas form when the immune system attempts to wall off substances that it perceives as foreign but is unable to eliminate. See, e.g., Divangahi (ed. 2013) The New Paradigm of Immunity to Tuberculosis (Advances in Experimental Medicine and Biology, book 783) Springer ISBN-10: 1461461103, ISBN-13: 978-1461461104. Such substances include infectious organisms such as mycobacteria, and are often sequestered by a layer of surrounding fibroblasts, which often form a calcified coating.

A “mycobacterial infection” is characterized by the detection of mycobacteria where it is unwanted, e.g., in a subject. Detection may be direct by microbiological or histological means, or by indirect means which may detect byproducts of cells, e.g., cell wall structures or components, or characteristic nucleic acids. In certain embodiments, the infection may be detected by symptoms, before more sensitive means are applied, or may prompt more sensitive detection efforts.

Unless otherwise indicated, a “biologic” is a molecule comprising amino acids in a polymer, generally linear, linked by peptide linkages into a protein. A polypeptide or protein may be conjugated to other entities, and will generally have at least 5, 10, 20, 30, 50 natural amino acids. Biologics can also include nucleic acids (RNA, DNA, aptamers), viral particles, and modified proteins (e.g., antibody fragments).

By proviso, the outer membrane acting biologic specifically excludes the fusion proteins described in WO2014001572 (and obvious homologs and analogs thereof), which describes a composition comprising two fusion proteins. In particular, the excluded composition is as follows:

(a) a first fusion protein comprising

(i) a first endolysin or a first domain, both having a first enzymatic activity, the enzymatic activity being at least one or more of the following: N-acetyl-b-D-muramidase (lysozyme, lytic transglycosylase), N-acetyl-b-D-glucosaminidase, N-acetylmuramoyl-L-alanine amidase, L-alanoyl-D-glutamate (LD) endopeptidase, c-D-glutamyl-meso-diaminopimelic acid (DL) peptidase, L-alanyl-D-iso-glutaminyl-meso-diaminopimelic acid (D-Ala-m-DAP) (DD) endopeptidase, or m-DAP-m-DAP (LD) endopeptidase;

(ii) at least one peptide stretch fused to the N- or C-terminus of the endolysin having the first enzymatic activity or the domain having the first enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and

(iii) a protein transduction domain (PTD) being at the N- or C-terminus of the first fusion protein, wherein the PTD is having the characteristic to deliver a cargo from the extracellular to the intracellular space of a cell; and

(b) a second fusion protein comprising

(i) a second endolysin or a second domain, both having a second enzymatic activity, the enzymatic activity being at least one or more of the following: lipolytic activity, cutinase, mycolarabinogalactanesterase, or alpha/beta hydrolase;

(ii) at least one peptide stretch fused to the N- or C-terminus of the endolysin having a second enzymatic activity or the domain having the second enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (iii) a protein transduction domain (PTD) being at the N- or C-terminus of the second fusion protein, wherein the PTD is having the characteristic to deliver a cargo from the extracellular to the intracellular space of a cell.

The biologic will typically be a single polypeptide, but may incorporate multiple polypeptide chains, which may be linked covalently or noncovalently, e.g., as a multiprotein complex. The separate peptides may be chemically conjugated to one another or to other molecules, often using non-peptide linkages, which would mean they are not fusion proteins. The polypeptide may have conjugations or other chemical modifications, e.g., methylations, glycosylations, acetylations, oxidations, etc. The term biologic does not depend upon how the entity is generated, as a chemically synthesized protein does not lose its biologic property by virtue of not being made completely by biological means.

A “chemotherapeutic” is a molecular structure which is a non-protein entity, generally to distinguish from natural or engineered proteins. Chemotherapeutics are typically described as “small molecules,” in contrast to typical protein structures. Thus, mycobacteria chemotherapeutics will typically be small molecule drugs, whose molecular sizes are smaller than standard proteins, e.g., smaller than proteins having molecular weights in the 10K, 15K, 20K, 25K, or 50K dalton size ranges. Examples of mycobacterial chemotherapeutics are antibiotics such as isoniazid, pyrazinamide, ethambutol, or rifampin, fluoroquinolones (e.g., ciprofloxacin, levefloxacin, moxifloxacin), cyclic peptides (e.g., capromycin, viomycin, enviomycin), thioamides (e.g., ethionamide, prothionamide), cycloserine, terizidone, an aminoglycoside, PAS, kanamycin, capreomycin, amikacin, streptomycin, microlide, a β-lactam, a β-lactamase inhibitor, clavulenic acid, trimethoprim, or sulfamethoxazole, clarithromycin, rifampicin, rifabutin, amikacin, azithromycin, or moxifloxacin, diarylquinoline, dedaquiline, TMC207, nitroimdazoles (including PA-824 and OPC-67683), oxazolidinones (including linezolid, sutezolid, and AZD5847), BTZ043, and SQ109.

An individual is “immunosuppressed” when the immune system is not as robust as a normal individual. The immunosuppression may be in humoral, cellular, or innate functions. The term is recognized in the clinical context, and often is induced by infection, treatment, or other environmental or health conditions.

An individual is HIV (or SIV) infected when infection is detectable, e.g., directly, indirectly, or symptomatically. For example, it will include one who has a detectable HIV type infection, which may be diagnosed by immunoassay or nucleic acid detection methods, by indirect methods of immune cell depletion, or by symptoms characteristic of the infection.

Additional symptoms of additional or progressed infection may exist, e.g., including cystic fibrosis, alpha-1 antitrypsin deficiency, Marfan's syndrome, primary ciliary dyskenesia, severe malnutrition, and other diagnoses or symptoms.

In various embodiments, the present disclosure can be applied to treatment of mammals, reptiles, amphibians, or fish. In particular, among the mammals will be primates (human and non-human), valuable livestock, marine or terrestrial mammals including orcas, dolphins, seals, walruses, tetrapods or bipeds such as zoo and exhibition animals such as elephants, camels, goats, sheep, cows, horses, and species designated or recognized as endangered. Among reptiles include snakes, crocodilians, tortoises, turtles, lizards, and tuataras. Amphibian subjects may include salamanders, frogs, and toads. Fish subjects will often be aquaculture subjects, but may be fish in exhibition aquaria, e.g., where admission is charged to view the fish.

The presently disclosed methods will also be applicable to infections characterized as environmental mycobacteria, atypical mycobacteria, Mycobacteria Other Than Tuberculosis (MOTT), and non-tuberculosis infections. See, e.g., Gangadharam (2012) Mycobacteria. I Basic Aspects Springer ASIN: BOOEZ16T9G; and Kazda, et al. (2009) The Ecology of Mycobacteria: Impact on Animal's and Human's Health Springer ISBN-10: 1402094124, ISBN-13: 978-1402094125.

In some embodiments, the present disclosure will be useful for the treatment of active infection, and more difficult treatment of latent infection. The latter are typically infections which are detectable but at low activity levels, e.g., where the infection comprises bacteria having minimal or only marginally detectable growth or replication activity. Many chemotherapeutic entities used for treatment are most effective on active infections, and often have limited therapeutic value, whether bacteristatic or bactericidal, for latent infections.

In certain embodiments, the mycobacteria infection will be extrapulmonary, e.g., having a focus of infection other than the more common lung site of infection. Other infections may include a lymphatic infection, or a disseminated infection, e.g., having diffuse foci which may be dispersed to locations beyond an initial primary infection site.

Other embodiments will be applicable to multidrug resistant infections, e.g., which are resistant to one or more of the drugs typically used for treatment. Typically, first line drugs are used, but the multidrug resistant infections may require more sophisticated or alternative drug combinations due to drug resistance or other evasion mechanisms. The problem is greater for extremely drug resistant infections, where the infection is resistant to many or most of the main drugs therapies typically used for treatment.

Some embodiments are applied to less common mycobacteria infections, e.g., those which lead to diagnoses of other diseases including Buruli ulcer, leprosy, abscesses, and others. See, e.g., Converse, et al. (2011) “Treating Mycobacterium ulcerans disease (Buruli ulcer): from surgery to antibiotics, is the pill mightier than the knife?” Future Microbiol. 6:1185-1198.

The detection of an infection may be accomplished by many methods of varying sensitivities. These may include, e.g., chest X-ray, microbiological culture, staining or culture from biopsy or sputum sample, histology evaluation, PCR or hybridization detection of nucleic acid, detection of mycobacteria products such as residual or released cell wall components, and Purified Protein Derivative or Mantoux test. Dinnes, et al. (2007) “A systematic review of rapid diagnostic tests for the detection of tuberculosis infection” Health Technol. Assess. 11:1-196.

The presently disclosed methods can be ascertained to have value by clinician evaluation methods, particularly evaluation of endpoint of efficacious therapy. Combination therapies will often lead to endpoints characterized as “cure” by achieving below some clinical threshold for sufficient duration, thus indicating low likelihood to reemerging active infection, or below certain clinically defined threshold of detection according to clinical standards. The combination treatments will often minimize amount of chemotherapeutic drugs needed, which may minimize significant side effects, or decrease the period of treatment, which will similarly decrease duration of those side effects. In other embodiments, the combination may allow certain chemotherapeutics to be eliminated from the treatment, which will decrease cost and side effects. And shorter duration will minimize cost of tracking the patient to ensure treatment compliance.

The presently disclosed combination therapies greatly reduce the average amount of mycobacteria chemotherapeutic administered (and in some cases eliminating certain components) to said subject in a time period, e.g., each week, thereby reducing side effects and cost of such drugs, or to reduce the duration of treatment with said mycobacteria chemotherapeutics, which can significantly reduce the need to track down and ensure patients are compliant with the treatment regimen.

D29 LysB is described as the LysB gene (GeneID:1261627/D29_12) and UniProtKB O64205 (VG12_BPMD2) annotated to be in InterPro IPR000675 (cutinase) and PFAm PF01083, with related sequences including generally phage derived forms of LysB, and such. These generally fall into the related classes of biologics, esterase, lipase, cutinase, and α/β hydrolase.

A “combination” package will typically package together a plurality of drugs or pills to be administered to the subject. These may be a combination of pills or therapeutic for administration substantially in a single visit with the subject, whether the subject comes to the health care provider, or the opposite. A plurality of therapeutic agents for the method may be provided in sealed card, sealed container; shrink wrap, or formulated capsules. In some embodiments, the drugs may be orally administered, or may include one or more injectable or inhalable. The health care provider will typically confirm that the subject has been dosed, and often provides some additional incentive to do so, as dosing may result in negative side effects which might appear worse than the inactive mycobacteria infection.

An “outer membrane acting” or “outer membrane permeabilizing” biologic typically is an enzymatic activity that increases the permeability of the mycobacteria outer membrane, e.g., according to the assays described. The entity will generally be an enzyme which acts on certain linkages which are important to maintain the permeability barrier which often prevents chemotherapeutics from reaching the target mycobacteria cells. Often the mycobacteria outer membrane acting entity will also possess a cell wall degrading activity that degrades, breaks down, disintegrates, or diminishes or reduces the integrity of a mycobacteria cell. See, e.g., Grover, et al. (2014) “Growth inhibition of Mycobacterium smegmatis by mycobacteriophage-derived enzymes” Enzym. Microb. Technol. 63:1-6. Reduction of integrity will often achieve an increase in permeability of the cell wall allowing small chemotherapeutics access across the cell wall permeability barrier. Thus, permeability may be a more sensitive assay than evaluation cell wall degradation.

The term “lytic” is typically used to mean “cell wall degrading”, partly because most (with certain exceptions) of the wall degrading catalytic activities are hydrolytic (Yang, et al. (2013) “Exposure to a Cutinase-like Serine Esterase Triggers Rapid Lysis of Multiple Mycobacterial Species” J. Biol. Chem. 288:382-392). Thus, much of the terminology used refers to “lytic” even if the catalytic mechanism does not involve hydrolysis. Among the possibilities of action on the cell wall or outer membrane are lipases, esterases, cutinases, and α/β hydrolases. Alternatively degradation of certain defined or artificial substrates may be useful assays for “lytic” or static activity (on a populational basis for the target). In this context, lytic may not necessarily imply that the cell lyses in response, though it may.

“Cell wall lytic activity” in a phage context is usually a characterization assigned to a structure based upon testing under artificial conditions, but such characterization can be specific for bacterial species, families, genera, or subclasses (which may be defined by sensitivity). Therefore, a “bacterium susceptible to a cell wall degrading activity” describes a bacterium whose cell wall is degraded, broken down, disintegrated, or that has its cell wall integrity diminished or reduced by a particular cell wall degrading activity or activities. Many other “lytic activities” originate from the host bacterial cells, and are important in cell division or phage release. Other phage derived cell wall degrading activities are found on the phage and have evolved to serve in various penetration steps of phage infection but would be physiologically abortive to phage replication if they kill the host cell before phage DNA is injected into the cell. The structures useful in the penetration steps are relevant in that these activities operate on normal hosts from the exterior. In some embodiments, the cell wall degrading activity is provided by an enzyme that is a non-holin enzyme and/or that is a non-lysin enzyme. In some embodiments, the cell binding activity is provided by an enzyme that is a non-holin enzyme and/or that is a non-lysin enzyme.

An “environment” of a bacterium can include an in vitro or an in vivo environment. In vitro environments are typically found in a reaction vessel, in some embodiments using isolated or purified bacteria, but can include surface sterilization, general treatment of equipment or animal quarters, or public health facilities such as water, septic, or sewer facilities. Other in vitro conditions may simulate mixed species populations, e.g., which include a number of symbiotically or interacting species in close proximity. Much of phage and bacterial study is performed in cultures in which the ratios of target host and phage are artificial and non-physiological. An in vivo environment preferably is in a host organism infected by the bacterium. In vivo environments include organs, such as bladder, kidney, lung, skin, heart and blood vessels, stomach, intestine, liver, brain or spinal cord, sensory organs, such as eyes, ears, nose, tongue, pancreas, spleen, thyroid, etc. In vivo environments include tissues, such as gums, nervous tissue, lymph tissue, glandular tissue, blood, sputum, etc., and may reflect cooperative interactions of different species whose survival may depend upon their interactions together. Catheter, implant, and monitoring or treatment devices which are introduced into the body may be sources of infection under normal usage. In vivo environments also may include the surface of food, e.g., fish, meat, or plant materials. Meats include, e.g., beef, pork, fish, chicken, turkey, quail, or other poultry. Plant materials include vegetable, fruits, or juices made from fruits and/or vegetables.

“Introducing” a composition to an environment includes administering a compound or composition, and contacting the bacterium with such. Introducing said compound or composition may often be effected by live bacteria which may produce or release such.

A “cell wall degrading protein” is a protein that has detectable, e.g., substantial, degrading activity on a cell wall or components thereof. Outer membrane acting biologics will be a subset where there is degradation of the outer membrane. “Lytic” activity may be an extreme form or result of the degrading activity. Exemplary bactericidal polypeptides include, e.g., the phage D29 LysB, structurally related entities, mutant and variants thereof, and other related constructs derived therefrom or from Mycobacterium phage Chy5, accession no-YP_008058282; Lysin B Mycobacterium phage L5 accession no-NP_039676; gp14 Mycobacterium phage Trixie accession no-AEL17844.1; serine esterase, cutinase M. smegmatis accession no-YP_890104.1.

Alternative phage derived degrading activities will be identified by their location on the phage tails or target host contact points of natural phage, mutated phase remnants (e.g., pyocins or bacteriocins), or encoded by prophage sequences. Preferred segments are derived, e.g., from mycobacteriophages, phages of Gram positive and Gram negative bacteria, genome sequence of M. tuberculosis, M. bovis BCG, M. smegmatis, atypical mycobacteria and MOTT.

A “LysB polypeptide” or grammatical variant thereof, refers to a bactericidal or bacteristatic activity encoded by the gene accession no NP_046827 from Pfam PF01083, or similar polypeptides. Exemplary variant LysB polypeptides include polypeptide polymorphic variants, alleles, mutants, and interspecies homologs that: (1) have an amino acid sequence that has greater than about 60% amino acid sequence identity, about 65%, 70%, 75%, 80%, 85%, 90%, preferably about 91%, 92%, 93%, 94%, 95%, 96%, 97%, 98% or 99% or greater amino acid sequence identity over one or more regions, e.g., of at least about 8, 12, 17, 25, 33, 50, 65, 80, 100, 200, or more amino acids, to an amino acid sequence encoded by a LysB nucleic acid from Mycobacterium phage Chy5, accession no-YP_008058282; Lysin B Mycobacterium phage L5 accession no-NP_039676; gp14 Mycobacterium phage Trixie accession no-AEL17844.1, or to an amino acid sequence of a muralytic polypeptide from serine esterase, cutinase M. Smegmatis accession no-YP_890104.1; (2) bind to antibodies, e.g., polyclonal antibodies, raised against a substantially purified immunogen comprising an amino acid sequence of an active fragment of LysB, and conservatively modified variants thereof (e.g., exhibit substantial immunogenicity); (3) specifically hybridize under stringent hybridization conditions to an anti-sense strand corresponding to a natural nucleic acid sequence encoding the LysB polypeptide, and conservatively modified variants thereof; (4) have a nucleic acid sequence that has greater than about 65%, 70%, 75%, 80%, 85%, 90%, or 95%, e.g., greater than about 96%, 97%, 98%, 99%, or higher nucleotide sequence identity over a region of at least about 25, 50, 100, 150, 200, 250, 300, 400, 500, 600, 700, etc., or more nucleotides, to the LysB encoding nucleic acid or a nucleic acid encoding fragment thereof. In some embodiments, segments are derived from the esterase domain. The presently described nucleic acids and proteins include both natural and recombinant molecules, the full length LysB polypeptide and fragments and variants thereof with permeability enhancing activity on cell wall components. Assays for permeability enhancing activity on cell wall components can be performed according to methods known to those of skill in the art, and as described herein.

In some embodiments, the LysB polypeptide has bactericidal activity alone against various mycobacteria strains, including, e.g., a M. tuberculosis, M. bovis, M. ulcerans, M. chelonae, M. marinum, M. avium complex, atypical mycobacteria, a mycobacteria other than tuberculosis (MOTT) species strain. Analogous measures of comparison may be applicable to other sequences, e.g., esterases, serine esterases, lipases, cutinases, phospholipases, carboxyesterases, and α/β hydrolases, as described herein.

Nucleic acids encoding mycobacteria outer membrane permeabilizing polypeptides can, in some embodiments, be amplified using PCR primers based on the sequence of described mycobacteria outer membrane permeabilizing polypeptides. For example, nucleic acids encoding LysB polypeptide variants and fragments thereof, as well as likely cell wall permeability acting candidates, can be amplified using primers. See, e.g., Vybiral, et al. (2003) FEMS Microbiol. Lett. 219:275-283. Thus, mycobacteria outer membrane acting polypeptides and fragments thereof include polypeptides that are encoded by nucleic acids that are amplified by PCR based on the sequence of the identified cell wall acting polypeptides. In a preferred embodiment, a bactericidal or bacteriostatic polypeptide or fragment thereof is encoded by a nucleic acid that is amplified by primers relevant to the LysB sequences described.

“GMP conditions” refers to good manufacturing practices, e.g., as defined by the Food and Drug Administration of the United States Government. Analogous practices and regulations exist in Europe, Japan, and most developed countries.

The term “substantially” in the above definitions of “substantially pure” generally means at least about 60%, at least about 70%, at least about 80%, or more preferably at least about 90%, and still more preferably at least about 95% pure, whether protein, nucleic acid, or other structural or other class of molecules.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acid analog refers to a compound that has the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain a basic chemical structure as a naturally occurring amino acid. Amino acid mimetic refers to a chemical compound that has a structure that is different from the general chemical structure of an amino acid, but that functions in a manner similar to a naturally occurring amino acid.

“Protein”, “polypeptide”, or “peptide” refers to a polymer in which a substantial fraction or all of the monomers are amino acids and are joined together through amide bonds, alternatively referred to as a polypeptide. When the amino acids are α-amino acids, either the L-optical isomer or the D-optical isomer can be used. Additionally, unnatural amino acids, e.g., β-alanine, phenylglycine, and homoarginine, are also included. Amino acids that are not gene-encoded may also be used in the presently disclosed compositions and methods. Furthermore, amino acids that have been modified to include appropriate structure or reactive groups may also be used. The amino acids can be D- or L-isomer, or mixtures thereof. L-isomers are generally preferred. Other peptidomimetics can also be used. For a general review, see, Spatola, in Weinstein, et al. (eds. 1983) Chemistry and Biochemistry of Amino Acids, Peptides and Proteins Marcel Dekker, New York, p. 267.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques. In particular, fusions of sequence may be generated, e.g., incorporating an upstream secretion cassette upstream of desired sequence to generate secreted protein product.

A “fusion protein” refers to a protein comprising amino acid sequences that are in addition to, in place of, less than, and/or different from the amino acid sequences encoding the original or native full-length protein or subsequences thereof. More than one additional domain can be added to a cell wall lytic protein as described herein, e.g., an epitope tag or purification tag, or multiple epitope tags or purification tags. Additional domains may be attached, e.g., which may add additional outer membrane acting activities (on the target or associated organisms of a mixed colony or biofilm), bacterial capsule degrading activities, targeting functions, or which affect physiological processes, e.g., vascular permeability. Alternatively, domains may be associated to result in physical affinity between different polypeptides to generate multichain polymer complexes.

The term “nucleic acid” refers to a deoxyribonucleotide, ribonucleotide, or mixed polymer in single- or double-stranded form, and, unless otherwise limited, encompasses known analogues of natural nucleotides that hybridize to nucleic acids in a manner similar to naturally occurring nucleotides. Unless otherwise indicated or by context, a particular nucleic acid sequence includes the complementary sequence thereof.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes typically include at least promoters and/or transcription termination signals. Typically, the recombinant expression cassette includes a nucleic acid to be transcribed (e.g., a nucleic acid encoding a desired polypeptide), and a promoter. Additional factors necessary or helpful in effecting expression may also be used, e.g., as described herein. In certain embodiments, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette. In certain embodiments, a recombinant expression cassette encoding an amino acid sequence comprising a lytic activity on a cell wall is expressed in a bacterial host cell.

A “heterologous sequence” or a “heterologous nucleic acid”, as used herein, is one that originates from a source foreign to the particular host cell, or, if from the same source, is modified from its original form. Modification of the heterologous sequence may occur, e.g., by treating the DNA with a restriction enzyme to generate a DNA fragment that is capable of being operably linked to the promoter. Techniques such as site-directed mutagenesis are also useful for modifying a heterologous sequence.

The term “isolated” refers to material that is substantially or essentially free from components which interfere with the activity of an enzyme or biologic. For a saccharide, protein, or nucleic acid as described herein, the term “isolated” refers to material that is substantially or essentially free from components which normally accompany the material as found in its native state. Typically, an isolated saccharide, protein, or nucleic acid is at least about 80% pure, usually at least about 90%, or at least about 95% pure as measured by band intensity on a silver stained gel or other method for determining purity. Purity or homogeneity can be indicated by a number of means well known in the art. For example, a protein or nucleic acid in a sample can be resolved by polyacrylamide gel electrophoresis, and then the protein or nucleic acid can be visualized by staining. For high resolution of the protein or nucleic, HPLC or a similar means for purification may be utilized.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The terms “identical” or percent “identity,” in the context of two or more nucleic acids or protein sequences, refer to two or more sequences or subsequences that are the same or have a specified percentage of amino acid residues or nucleotides that are the same, when compared and aligned for maximum correspondence, as measured using one of the sequence comparison algorithms or by visual inspection.

The phrase “substantially identical,” in the context of two nucleic acids or proteins, refers to two or more sequences or subsequences that have, over the appropriate segment, at least greater than about 60% nucleic acid or amino acid sequence identity, 65%, 70%, 75%, 80%, 85%, 90%, preferably 91%, 92%, 93%, 94%, 95%, 96%, 97%, 98% or 99% nucleotide or amino acid residue identity, when compared and aligned for maximum correspondence, as measured using one of the following sequence comparison algorithms or by visual inspection. Preferably, the substantial identity exists over a region of the sequences that corresponds to at least about 13, 15, 17, 23, 27, 31, 35, 40, 50, or more amino acid residues in length, more preferably over a region of at least about 100 residues, and most preferably the sequences are substantially identical over at least about 150 residues. Longer corresponding nucleic acid lengths are intended, though codon redundancy may be considered. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Nat'l Acad. Sci. USA 85:2444, by computerized implementations of these and related algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, Ausubel, et al., eds., Current Protocols, a joint venture between Greene Publishing Associates, Inc. and John Wiley & Sons, Inc. (1995 and Supplements) (Ausubel)).

Examples of algorithms that are suitable for determining percent sequence identity and sequence similarity are the BLAST and BLAST 2.0 algorithms, which are described in Altschul, et al. (1990) J. Mol. Biol. 215:403-410 and Altschuel, et al. (1977) Nucleic Acids Res. 25:3389-3402, respectively. Software for performing BLAST analyses is publicly available through the National Center for Biotechnology Information (ncbi.nlm.nih.gov/) or similar sources. This algorithm involves first identifying high scoring sequence pairs (HSPs) by identifying short “words” of length W in the query sequence, which either match or satisfy some positive-valued threshold score T when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff (1989) Proc. Nat'l Acad. Sci. USA 89:10915).

In addition to calculating percent sequence identity, the BLAST algorithm also performs a statistical analysis of the similarity between two sequences (see, e.g., Karlin and Altschul (1993) Proc. Nat'l Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or proteins are substantially identical is that the protein encoded by the first nucleic acid is immunologically cross reactive with the protein encoded by the second nucleic acid, as described below. Thus, a protein is typically substantially identical to a second protein, for example, where the two peptides differ only by conservative substitutions. Another indication that two nucleic acid sequences are substantially identical is that the two molecules hybridize to each other under stringent conditions, as described below.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 15° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and, the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is typically at least two times background, preferably 10 times background hybridization. Exemplary stringent hybridization conditions can be as following: 50% formamide, 5.times.SSC, and 1% SDS, incubating at 42° C., or, 5.times.SSC, 1% SDS, incubating at 65° C., with wash in 0.2.times.SSC, and 0.1% SDS at 65° C. For PCR, a temperature of about 36° C. is typical for low stringency amplification, although annealing temperatures may vary between about 32-48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90-95. ° C. for 30-120 sec, an annealing phase lasting 30-120 sec, and an extension phase of about 72° C. for 1-2 min. Protocols and guidelines for low and high stringency amplification reactions are available, e.g., in Innis, et al. (1990) PCR Protocols: A Guide to Methods and Applications Academic Press, N.Y.

The phrases “specifically binds to a protein” or “specifically immunoreactive with”, when referring to an antibody refers to a binding reaction which is determinative of the presence of the protein in the presence of a heterogeneous population of proteins and other biologics. Thus, under designated immunoassay conditions, the specified antibodies bind preferentially to a particular protein and do not bind in a significant amount to other proteins present in the sample. Specific binding to a protein under such conditions requires an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual Cold Spring Harbor Publications, New York, for a description of immunoassay formats and conditions that can be used to determine specific immunoreactivity.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given protein. For instance, the codons CGU, CGC, CGA, CGG, AGA, and AGG all encode the amino acid arginine. Thus, at each position where an arginine is specified by a codon, the codon can be altered to another of the corresponding codons described without altering the encoded protein. Such nucleic acid variations are “silent variations,” which are one species of “conservatively modified variations.” Each polynucleotide sequence described herein which encodes a protein also describes possible silent variations, except where otherwise noted. One of skill will recognize that each codon in a nucleic acid (except AUG, which is ordinarily the only codon for methionine, and UGG which is ordinarily the only codon for tryptophan) can be modified to yield a functionally identical molecule by standard techniques. Accordingly, each “silent variation” of a nucleic acid which encodes a protein is typically implicit in each described sequence.

Those of skill recognize that many amino acids can be substituted for one another in a protein without affecting the function of the protein, e.g., a conservative substitution can be the basis of a conservatively modified variant of a protein such as the disclosed cell wall lytic proteins. An incomplete list of conservative amino acid substitutions follows. The following eight groups each contain amino acids that are normally conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V), Alanine (A); 6) Phenylalanine (F), Tyrosine (Y), Tryptophan (W); 7) Serine (S), Threonine (T), Cysteine (C); and 8) Cysteine (C), Methionine (M) (see, e.g., Creighton (1984) Proteins).

Furthermore, one of skill will recognize that individual substitutions, deletions, or additions which alter, add, or delete a single amino acid or a small percentage of amino acids (typically less than 5%, more typically less than 1%) in an encoded sequence are effectively “conservatively modified variations” where the alterations result in the substitution of an amino acid with a chemically similar amino acid. Conservative substitution tables providing functionally similar amino acids are well known in the art.

One of skill will appreciate that many conservative variations of proteins, e.g., cell wall permeabilizing proteins, and nucleic acids which encode proteins yield essentially identical products. For example, due to the degeneracy of the genetic code, “silent substitutions” (e.g., substitutions of a nucleic acid sequence which do not result in an alteration in an encoded protein) are an implied feature of each nucleic acid sequence which encodes an amino acid. As described herein, sequences are preferably optimized for expression in a particular host cell used to produce the outer membrane acting biologics (e.g., yeast, human, and the like). Similarly, “conservative amino acid substitutions,” in one or a few amino acids in an amino acid sequence are substituted with different amino acids with highly similar properties, are also readily identified as being highly similar to a particular amino acid sequence, or to a particular nucleic acid sequence which encodes an amino acid. Conservatively substituted variations of any particular sequence included in the presently disclosed compositions and methods. See also, Creighton (1984) Proteins Freeman and Company. In addition, individual substitutions, deletions or additions which alter, add or delete a single amino acid or a small percentage of amino acids in an encoded sequence generally are also “conservatively modified variations”.

The presently disclosed compositions and methods can involve the construction of recombinant nucleic acids and the expression of genes in host cells, e.g., bacterial host cells. Optimized codon usage for a specific host will often be applicable. Molecular cloning techniques to achieve these ends are known in the art. A wide variety of cloning and in vitro amplification methods suitable for the construction of recombinant nucleic acids such as expression vectors are well known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); and Current Protocols in Molecular Biology, Ausubel, et al., eds., Current Protocols, a joint venture between Greene Publishing Associates, Inc. and. John Wiley & Sons, Inc., (1999 Supplement) (Ausubel). Suitable host cells for expression of the recombinant polypeptides are known to those of skill in the art, and include, for example, prokaryotic cells, such as E. coli, and eukaryotic cells including insect, mammalian, and fungal cells (e.g., Aspergillus niger).

Examples of protocols sufficient to direct persons of skill through in vitro amplification methods, including the polymerase chain reaction (PCR), the ligase chain reaction (LCR), Q.beta.-replicase amplification and other RNA polymerase mediated techniques are found in Berger, Sambrook, and Ausubel, as well as Mullis, et al. (1987) U.S. Pat. No. 4,683,202; PCR Protocols A Guide to Methods and Applications (Innis, et al. eds.) Academic Press Inc. San Diego, Calif. (1990) (Innis); Arnheim and Levinson (Oct. 1, 1990) C&EN 36-47; The Journal Of NIH Research (1991) 3:81-94; (Kwoh, et al. (1989) Proc. Nat'l Acad. Sci. USA 86:1173; Guatelli, et al. (1990) Proc. Nat'l Acad. Sci. USA 87:1874; Lomell, et al. (1989) J. Clin. Chem. 35:1826; Landegren, et al. (1988) Science 241:1077-1080; Van Brunt (1990) Biotechnology 8:291-294; Wu and Wallace (1989) Gene 4:560; and Barringer, et al. (1990) Gene 89:117. Improved methods of cloning in vitro amplified nucleic acids are described in Wallace, et al., U.S. Pat. No. 5,426,039.

### IV. Permeability Acting Biologics

The presently disclosed compositions and methods are based partly upon the recognition that certain permeability boundaries prevent the access of mycobacteria chemotherapeutics to reach their proper site of action. In particular, for individual cells, the mycobacteria outer membrane is a permeability barrier, with waxy lipid properties which may protect the mycobacteria from the chemotherapeutic. A second barrier can be formed by a macrophage which has encapsulated the mycobacteria, and sequestered it into an intracellular compartment, e.g., a lysosome. The mycobacteria may not be killed there, but may lay dormant and viable to become active under the right circumstances. As such, the mycobacteria may be virtually inert, and the cell less susceptible to very low levels of chemotherapeutic which reach it from the macrophage cell barriers. A third barrier can be formed by a granuloma, which may form around the mycobacterial cell. The granuloma can contain many macrophages, directed to eliminate encapsulated mycobacteria, and can include free mycobacteria which have escaped the macrophages. The granuloma often puts up a barrier to sequester the macrophages and other cells, within a fibroblast boundary. A calcified layer may also be put up to further isolate the granuloma contents from the surrounding tissue.

A. Mycobacteria Outer Membrane Acting Biologics

Biologics which will act on the known structural components making up the mycobacteria outer membrane will typically cleave the bond between arabinogalactan and mycolates, trehalose and mycolates, arabinogalactin and peptidoglycan linkages therein. These activities will typically be found in the categories of esterases, lipases, cutinases, and α/β hydrolases. Payne and Hatfull (2012) “Mycobacteriophage Endolysins: Diverse and Modular Enzymes with Multiple Catalytic Activities” PLoS ONE 7:e34052; Podobnik, et al. (2009) “A Mycobacterial Cyclic AMP Phosphodiesterase That Moonlights as a Modifier of Cell Wall Permeability” J. Biol. Chem. 284:32846-32857; Lopez-Marin (2012) “Nonprotein Structures from Mycobacteria: Emerging Actors for Tuberculosis Control” Clinical and Developmental Immunology 2012:ID 917860; and Dedieu, et al. (2013) “Mycobacterial lipolytic enzymes: A gold mine for tuberculosis research” Biochimie 95:66e73. Thus, many of the members of these groups have activities that can be used in the presently disclosed compositions and methods.

Among the esterase proteins, preferred embodiments will be found in the following Pfam number, identification description, description: PF01095, Pectinesterase, Pectinesterase; PF01339, CheB_methylest, CheB methylesterase; PF03009, GDPD, Glycerophosphoryl diester phosphodiesterase family; PF04043, PMEI, Plant invertase/pectin methylesterase inhibitor; PF00135, COesterase, Carboxylesterase family; PF00149, Metallophos, Calcineurin-like phosphoesterase; PF00756, Esterase, Putative esterase; PF00975, Thioesterase, Thioesterase domain; PF03996, Hema_esterase, Hemagglutinin esterase; PF12850, Metallophos_2, Calcineurin-like phosphoesterase superfamily domain; PF03283, PAE, Pectinacetylesterase; PF05057, DUF676, Putative serine esterase (DUF676); PF10503, Esterase_phd, Esterase PHB depolymerase; PF13449, Phytase-like, Esterase-like activity of phytase; PF01083, Cutinase, Cutinase; PF00326, Peptidase_S9, Prolyl oligopeptidase family; PF12695, Abhydrolase_5, Alpha/beta hydrolase family; PF00082, Peptidase_S8, Subtilase family; PF00150, Cellulase, Cellulase (glycosyl hydrolase family 5); PF00187, Chitin_bind_1, Chitin recognition protein; PF00331, Glyco_hydro_10, Glycosyl hydrolase family 10; PF00457, Glyco_hydro_11, Glycosyl hydrolases family 11; and PF01425, Amidase, Amidase.

Among the lipase proteins, preferred embodiments will be found in the following Pfam number, identification description, description: Pfam no., ID, Description; PF00388, PI-PLC-X, Phosphatidylinositol-specific phospholipase C, X domain; PF01764, Lipase_3, Lipase (class 3); PF00657, Lipase_GDSL, GDSL-like Lipase/Acylhydrolase; PF04083, Abhydro_lipase, Partial alpha/beta-hydrolase lipase region; PF13472, Lipase_GDSL_2, GDSL-like Lipase/Acylhydrolase family; PF03583, LIP, Secretory lipase; PF01734, Patatin, Patatin-like phospholipase; PF02230, Abhydrolase_2, Phospholipase/Carboxylesterase; PF03490, Varsurf_PPLC, Variant-surface-glycoprotein phospholipase C; PF12146, Hydrolase_4, Putative lysophospholipase; PF00068, Phospholip_A2_1, Phospholipase A2; PF05778, Apo-CIII, Apolipoprotein CIII (Apo-CIII); PF10081, Abhydrolase_9, Alpha/beta-hydrolase family; PF01083, Cutinase, Cutinase; PF05057; DUF676, Putative serine esterase (DUF676); and PF00005, ABC_tran, ABC transporter. See, e.g., Arpigny and Jaeger (1999) “Bacterial lipolytic enzymes: classification and properties” Biochem. J. 343:177-183; and Grover, et al. (2014) Enzyme Microb. Technol. 63:1-6.

Among the cutinase proteins, preferred embodiments will be found in the following Pfam number, identification description, description: PF01083, Cutinase, Cutinase; PF12695, Abhydrolase_5, Alpha/beta hydrolase family; PF00076, RRM_1 or RNA, recognition motif (a.k.a. RRM or RBD or RNP domain); PF00096, zf-C2H2 or Zinc, finger or C2H2 type; PF00172, Zn_clus, Fungal Zn(2)-Cys(6) binuclear cluster domain; PF00320, GATA, GATA zinc finger; PF08237, PE-PPE, PE-PPE domain; PF09659, Cas_Csm6, CRISPR-associated protein (Cas_Csm6); PF13426, PAS_9 PAS domain; PF13465, zf-H2C2_2, Zinc-finger double domain; and PF14259, RRM_6, RNA recognition motif (a.k.a. RRM, RBD, or RNP domain).

Among the α/β hydrolase proteins, preferred embodiments will be found in the following Pfam number, identification description, description: PF12697, Abhydrolase_6, Alpha/beta hydrolase family; PF00561, Abhydrolase_1, alpha/beta hydrolase fold; PF02230, Abhydrolase_2, Phospholipase/Carboxylesterase; PF12146, Hydrolase_4, Putative lysophospholipase; and PF00702, Hydrolase, haloacid dehalogenase-like hydrolase.

Selecting among the lysB based activity, similar sequences, e.g., which have high sequence matching, are screened for activity, as described below. Thus, alternative embodiments of esterase type biologics can be selected, by example in PF01083, as follows: Protein, Source, Accession no., % ID; LysB, Mycobacterium Phage D29 phage, NP_046827, 100; lysin B, Mycobacterium phage Chy5, YP_008058282, 99; lysinB, Mycobacterium phage SWU1, YP_006382930, 91; Lysin B, Mycobacterium phage L5, NP_039676, 90; gp14, Mycobacterium phage Trixie, AEL17844.1, 77; lysin B, Mycobacterium phage Echild, YP_009004460.1, 77; Cutinase, M. Smeg, YP_885788.1, 36; serine esterase, cutinase, M. Smeg, YP_890104.1, 36; NUDIX hydrolase, M. Smeg, YP_887512.1, 34; Probable cutinase, Cut5b, Mycobacterium tuberculosis H37Rv, YP_178008.1, 26; hypothetical protein NECHADRAFT_8492, Nectria haematococca mp, XP_003045063.1, 31. Above some sequence identity, most will preserve the function, and below will retain function at a lower probability. Below some other measure of identity, most will have lesser probability of sharing function, but above will typically have greater. Those with highest identity measures might be expected to have greatest likelihood of similar function. However, diversity from natural sources have been subjected to selection, so the distribution of disparity may be focused on noncritical parts of the protein. By looking carefully at sequence alignments, it may be possible to recognize critical functional motifs, which may lead to more accurate sequence evaluation for sequences likely to retain function.

With a biologic having detectable function, the sensitivity of function to changes can be evaluated. The boundaries of the function may be evaluated by truncation constructs removing segments from the N and C terminus of the sequence. Mutagenesis analyses can evaluate where and how sensitive the function is to conservative or other substitutions. Methods for such are: well known in the art, and are described in the references listed herein.

Within each category of function, the structural motifs which are characteristic of a function may be evaluated and identified. Such motifs may be used to screen sequence databases for additional biologics which may exhibit the desired functions.

### Permeability Assays

Permeability assays across the mycobacteria outer membrane can be based upon outside in or inside out. For example, the assay may be designed to detect when a label reaches the cell surface from the extracellular milieu. Conversely, the cells may normally contain or be loaded with indicator, e.g., in the periplasmic space, and release to the extracellular milieu may be evaluated. Details of the kinetics of indicator passive leakage will need to the determined, and the conditions of assay must be compatible with biologic activity of the tested entity. Often different concentrations of biologic are evaluated. The physiological state of the target strain should be carefully monitored to ensure that linkages targeted by the biologic are present in forms comparable to natural infections.

Assays to monitor how quickly cells can be loaded with indicator, which would reflect permeability of the mycobacteria outer membrane, may use a dye or indicator which changes color upon reaching the periplasmic space. The periplasmic space typically has a different pH or oxidation state than outside of the cell, and the kinetics of indicator reaching that location may be monitored over time upon exposure of the cells to the outer membrane acting biologic. Biologics having high activity will typically allow more indicator past the barrier than biologics having lower activity. Similarly, larger amounts of entities having a set amount of activity will generally allow more indicator to reach the periplasmic space than lesser amounts.

Conversely, assays may be developed which evaluate the rate of leakage of indicators from the periplasmic space to the external milieu. In some embodiments, the indicator will be a dye which is taken up into the periplasmic space, while in other embodiments, certain entities which normally accumulate in the periplasmic space may traced. Often the target cell may be recombinantly generated to produce a traceable indicator into the periplasmic space. The cell may be loaded up with indicator, then washed so free indicator is removed unless it is intimately associated, e.g., inside the mycobacteria outer membrane. Preferably leakage is slow, unless the permeability barrier is compromised. The ability of test biologics to cause release can be the basis for evaluating activity of the biologics to compromise the mycobacteria outer membrane barrier.

Assays may be developed to be performed on plates, which provide a spatial separability. Other assays may be in solution, and may be developed with microfluidic strategies for high throughput evaluation. Fluorescent cell sorting technologies can be easily applied with such formats.

Both assay methods, evaluating permeability from outside to in, or inside to out, can be developed into larger scale assays. These may be developed into more qualitative than quantitative, which may be useful when false positive signals are more problematic than false negatives. With higher throughput assays, testing of ten, hundreds, thousands, or more candidates can be performed simultaneously in parallel. With high throughput, the methodology can be used to evaluate larger scale screening efforts, e.g., of mutagenesis efforts using random mutagenesis, to find entities with the preferred or optimal properties. Moreover, large scale efforts may allow for easier screening of large genetic data sources to test many different alternative sequences expressed in different conditions of growth for expression.

Such screening methods allow for application of the screening on large scales. Gene shuffling strategies can be used to generate products for testing and screening for the desired mycobacteria outer membrane permeability, macrophage permeability, or granuloma permeability activity.

B. Macrophage and Granuloma Permeability Acting Biologics

Biologics which may affect either or both of the macrophage or fibroblast permeability barriers start with peptides known to have cell penetrating activity. These cell penetrating peptides (CPP) often can transport associated cargo, which may be attached peptides or fusion peptides. One example of such is the Mtb Mce3 protein. Other potential candidates may affect efflux mechanisms of the eukaryotic cells making up granuloma outer coverings (barriers), and would include efflux inhibitors or drugs such as analogs of reserpine (see, e.g., Pearce, et al. (1990) “Structural characteristics of compounds that modulate P-glycoprotein-associated multidrug resistance” Adv. Enzyme Regul. 30:357-73; and Pearce, et al. (1989) “Essential features of the P-glycoprotein pharmacophore as defined by a series of reserpine analogs that modulate multidrug resistance” Proc. Natl. Acad. Sci. USA 86:5128-32), of verapamil (see, e.g., Toffoli, et al. (1995) “Structure-activity relationship of verapamil analogs and reversal of multidrug resistance” Biochem. Pharmacol. 50:1245-55; and Pirker, et al. (1990) “Reversal of multi-drug resistance in human KB cell lines by structural analogs of verapamil” Int. J. Cancer 45:916-9), and functional and/or structural analogs and derivatives (e.g., rauwolfia alkaloids and Ca⁺⁺ channel blockers, see Merck Index, Canadian Royal Society of Chemistry). See, e.g., Guirado and Schlesinger (2013) “Modeling the Mycobacterium tuberculosis Granuloma—the Critical Battlefield in Host Immunity and Disease” Frontiers in Immunology 4:98; Harding, et al. (2011) “Granuloma transplantation: an approach to study mycobacterium-host interactions” Frontiers in Microbiology 2(art 245):1-10; Viveiros, et al. (2012) “Inhibitors of mycobacterial efflux pumps as potential boosters for anti-tubercular drugs” Expert Reviews in Anti Infective Therapy 10:983-98; Seral, et al. (2003) “Influence of P-glycoprotein and MRP efflux pump inhibitors on the intracellular activity of azithromycin and ciprofloxacin in macrophages infected by Listeria monocytogenes or Staphylococcus aureus” Journal of Antimicrobial Chemotherapy 51:1167-73; Adams, et al. (2014) “Verapamil, and its metabolite norverapamil, inhibit macrophage-induced, bacterial efflux pump-mediated tolerance to multiple anti-tubercular drugs” Journal of Infectious Disease 210:456-66; Gupta, et al. (2013) “Acceleration of tuberculosis treatment by adjunctive therapy with verapamil as an efflux inhibitor” American Journal of Respiratory and Critical Care Medicine 188:600-7; Balganesh, et al. (2012) “Efflux pumps of Mycobacterium tuberculosis play a significant role in antituberculosis activity of potential drug candidates” Antimicrobial Agents and Chemotherapy 56:2643-51; Adams, et al. (2011) “Drug tolerance in replicating mycobacteria mediated by a macrophage-induced efflux mechanism” Cell 145:39-53; Martins, et al. (2008) “Inhibitors of Ca2+ and K+ transport enhance intracellular killing of M. tuberculosis by non-killing macrophages” In Vivo 22:69-75; Amaral, et al. (2007) “Enhanced killing of intracellular multidrug-resistant Mycobacterium tuberculosis by compounds that affect the activity of efflux pumps” Journal of Antimicrobial Chemotherapy 59:1237-46; and Rey-Jurado, et al. (2013) “Activity and interactions of levofloxacin, linezolid, ethambutol and amikacin in three-drug combinations against Mycobacterium tuberculosis isolates in a human macrophage model” International Journal of Antimicrobial Agents 42:524-30. Screens for such efflux inhibitors can be readily developed, e.g., screening for the presence of an efflux inhibitor which affects accumulation of a labeled (e.g., fluorescence or other) transported molecule inside the cell. See, e.g., Ansbro, et al. (2013) “Screening compounds with a novel high-throughput ABCB1-mediated efflux assay identifies drugs with known therapeutic targets at risk for multidrug resistance interference” PLoS One 8:e60334; and Kourtesi, et al. (2013) “Microbial efflux systems and inhibitors: approaches to drug discovery and the challenge of clinical implementation” Open Microbiology Journal 7:34-52.

Other proteins which are likely to have similar capability will be those in the related Pfam, and include, e.g., Pfam no., GeneID, description; PF02470, MCE, mce related protein; PF00493, MCM, MCM2/3/5 family; PF01331, mRNA_cap_enzyme, mRNA capping enzyme or catalytic domain; PF03919, mRNA_cap_C, mRNA capping enzyme or C-terminal domain; PF04075, DUF385, Domain of unknown function (DUF385); PF06271, RDD, RDD family; PF07686, V-set, Immunoglobulin V-set domain; and PF11887, DUF3407, Protein of unknown function (DUF3407); PF13669, Glyoxalase_4, Glyoxalase/Bleomycin resistance protein/Dioxygenase superfamily.

Proteins from non-mycobacteria sources which have CPP features include, e.g., (Proteins, Pfam no.); Tat, PF00539; Penetratin, PF04280; Transportan, none; MPG, PF02245; Pep-1, PF04512; MAP, PF02141; SAP, PF02037; hCT, PF03390; and SynB, PF04099.

Analogous permeability assays may be developed for macrophage permeability to reach the engulfed mycobacteria, or across the granuloma boundary separating the external from interior. Again, these may be inside to out, or outside to in. Assays to detect biologics (or chemotherapeutics) which affect the separation can be applied.

Indicators which change upon reaching the lysosomes should be easy to identify, as the lysosomes have both peculiar pH and oxidation states. Macrophages or monocytes may be exposed to such indicators, and tested to evaluate whether potential biologics affect uptake from outside the cell. The Cell Permeability Peptides are named because they do induce uptake. Again, with any positive activity, sequences being classified into similar categories are likely candidates for further testing for similar functions.

In some embodiments, cells other than macrophages may be used to screen for biologics which affect cell uptake of extracellular indicators. Preferably cells with many properties in common with macrophages, monocytes, or fibroblasts are used, and can be from human, primate, or other species. Many of the permeability modulating biologics would not be species specific.

For assays on permeability for granulomas, in vitro models have been described. Kapoor, et al. (2013) “Human Granuloma In Vitro Model, for TB Dormancy and Resuscitation” PLoS ONE 8:e53657. Permeability assays can be developed, both from outside in, and inside out. Many of the same biologics may affect the granuloma fibroblast barrier, as the macrophage barriers described above. In vivo animal models may be developed to evaluate when synergy can be detected. See, e.g., Hoff, et al. (2011) “Location of Intra- and Extracellular M. tuberculosis Populations in Lungs of Mice and Guinea Pigs during Disease Progression and after Drug Treatment” PLoS ONE 6(3):e17550.

In another embodiment, a biofilm acting activity may be used in the combination. See, e.g., Chan and Abedon (2014) “Bacteriophages and their Enzymes in Biofilm Control” Current Pharmaceutical Design September 5 Epub; Parasion, et al. (2014) “Bacteriophages as an alternative strategy for fighting biofilm development” Polish Journal of Microbiology 63:137-45; Richards and Melander (2009) “Controlling bacterial biofilms” Chembiochem. 10:2287-94; Donlan (2009) “Preventing biofilms of clinically relevant organisms using bacteriophage” Trends in Microbiology 17:66-72; Islam, et al. (2012) “Targeting drug tolerance in mycobacteria: a perspective from mycobacterial biofilms” Expert Reviews in Anti Infective Therapy 10:1055-66; Ishida, et al. (2011) “Inhibitory effect of cyclic trihydroxamate siderophore, desferrioxamine E, on the biofilm formation of Mycobacterium species” in Ishida, et al. (2011) Biological and Pharmaceutical Bulletin 34:917-20; Ojha and Hatfull (2007) “The role of iron in Mycobacterium smegmatis biofilm formation: the exochelin siderophore is essential in limiting iron conditions for biofilm formation but not for planktonic growth” Molecular Microbiology 66:468-83; Ojha, et al. (2010) “Enzymatic hydrolysis of trehalose dimycolate releases free mycolic acids during mycobacterial growth in biofilms” Journal of Biological Chemistry 285:17380-9; Ojha, et al. (2008) “Growth of Mycobacterium tuberculosis biofilms containing free mycolic acids and harbouring drug-tolerant bacteria” Molecular Microbiology 69:164-74; and Islam, et al. (2013) “Antimycobacterial efficacy of silver nanoparticles as deposited on porous membrane filters” Material Science and Engineering C: Materials for Biological Applications 33:4575-81.

In certain embodiments, the macrophage or fibroblast permeability biologics have a transport component, which will cause uptake of the biologic and “cargo” physically attached. As such, in certain embodiments, the biologic is physically attached to other components to be targeted to the mycobacteria. These may be by peptide linkages, e.g., fusion proteins, by non-peptide conjugation chemistry, or by non-covalent conjugation association.

Chemical linkages or bioconjugation technologies may be used. See, e.g., Niemeyer (ed. 2010) Bioconjugation Protocols: Strategies and Methods (Methods in Molecular Biology) Humana Press; Hermanson (2008) Bioconjugate Techniques (2d ed.) Academic Press; Lahann (ed. 2009) Click Chemistry for Biotechnology and Materials Science Wiley; Rabuka (2010) “Chemoenzymatic methods for site-specific protein modification” Curr. Opin. Chem. Biol. 14:790-96. Epub 2010 Oct. 26; Tiefenbrunn and Dawson (2010) “Chemoselective ligation techniques: modern applications of time-honored chemistry” Biopolymers 94:95-106; Nwe and Brechbiel (2009) “Growing applications of “click chemistry” for bioconjugation in contemporary biomedical research” Cancer Biother Radiopharm. 24:289-302; de Graaf, et al. (2009) “Nonnatural amino acids for site-specific protein conjugation” Bioconjug Chem. 20:1281-95; the journal Bioconjugate Chemistry (ACS); and Thordarson, et al. (2006) “Well-defined protein—polymer conjugates—synthesis and potential applications” Applied Microbiology and Biotechnology 73:243-254, DOI: 10.1007/s00253-006-0574-4. For example, specific amino acids can be incorporated or added at either end, perhaps to constructs which have removed non-critical like residues, e.g., for cysteine residues. Accessible cysteine residues can be used to connect the segments by disulfide linkages. Cysteine residues can also be linked with bifunctional maleimide linkers with thioether bonds. The linkers can also have a hydrocarbon spacer of appropriate length, e.g., 6, 9, 12, 15, 18, 21, 25, 29, 35, or more carbon chains.

Non-covalent conjugation may include high affinity association, e.g., avidin-streptavidin, avidin-biotin, lectin-carbohydrate interactions, etc. See, e.g., Life Technologies catalog.

As described below, often the methods used refer to use directed to the mycobacteria outer membrane acting biologic, but often similar methods and strategies may be applied to screening for entities which can affect the access of chemotherapeutics to mycobacteria localized inside of macrophages or monocytes, e.g., within the lysosomes of the host cells. Likewise, other granuloma permeability enzymes or biologics may be screened for or characterized using permeability assays directed to compromising the barrier, typically fibroblasts, to chemotherapeutics reaching inside a granuloma.

### VII. Commercial Applications

Various applications of the described methods can be immediately recognized. One important application is as antimycobacterial treatment of articles which may be contaminated in normal use. Locations, equipment, environments, or the like where target mycobacteria may be public health hazards may be treated using such entities. Locations of interest include public health facilities where the purpose or opportunity exists to deal with target mycobacteria containing materials. These materials may include waste products, e.g., liquid, solid, or air. Aqueous waste treatment plants may incorporate such to eliminate the target from effluent, whether by treatment with the enzyme entities directly, or by release of cells which produce such. Solid waste sites may introduce such to minimize possibility of target host outbreaks. Conversely, food preparation areas or equipment need to be regularly cleaned, and the presently disclosed compositions and methods can effectively eliminate target bacteria. Medical and other public environments subject to contamination may warrant similar means to minimize growth and spread of target microorganisms. The methods may be used in contexts where sterilization elimination of target bacteria is desired, including air filtration systems for an intensive care unit.

Alternative applications include use in a veterinary or medical context. Means to determine the presence of particular mycobacteria, or to identify specific targets may utilize the effect of selective agents on the population or culture. Inclusion of bacteriostatic or bactericidal activities to cleaning agents, including washing of animals and pets, may be desired.

The LysB and related biologics can be used to treat mycobacteria infections of, e.g., humans or animals, alone or in combination with mycobacteria chemotherapeutics. These biologics can be administered alone or in combination with additional chemotherapeutics or can be administered to a subject that has contracted a mycobacteria infection in the methods described. In some embodiments, LysB biologics are used with chemotherapeutics to treat infections caused by mycobacteria that replicate slowly as the killing mechanism does not depend so much upon host cell replication. Many antibacterial agents, e.g., antibiotics, are most useful against replicating bacteria. Bacteria that replicate slowly have doubling times of, e.g., about 1-72 hours or more, 1-48 hours, 1-24 hours, 1-12 hours, 1-6 hours, 1-3 hours, or 1-2 hours. Different types may have different susceptibilities to the combinations.

In some embodiments, these biologics are used to treat humans or other animals that are infected with a mycobacteria species. In some embodiments, the LysB or other mycobacteria outer membrane acting biologics are used, alone or in combination with other mycobacteria chemotherapeutics, to treat humans or other animals that are infected with a mycobacteria species. In other embodiments, the combination may also be administered with other treatments, which may include other features of the typical treatment for the infections, e.g., with one or more features of typical standard of care. See, e.g., for tuberculosis, World Health Organization Geneva: Treatment of Tuberculosis: Guidelines (4th Ed. 2010) ISBN 13:9789241547833; Revised National Tuberculosis Control Programme (DOTS-Plus Guidelines) (2010) Central TB Division, Directorate General of Health Services, Ministry of Health & Family Welfare, Nirman Bhavan, New Delhi—110011; Tuberculosis Coalition for Technical Assistance (2007) Handbook for Using the International Standards for Tuberculosis Care, Tuberculosis Coalition for Technical Assistance, The Hague; TB CARE (2014) I. International Standards for Tuberculosis Care (Edition 3) TB CARE I, The Hague; Norton and Holland (2012)“Current management options for latent tuberculosis: a review” Infection and Drug Resistance 5:163-73; Centers for Disease Control and Prevention (CDC) (1998) Prevention and treatment of tuberculosis among patients infected with human immunodeficiency virus: principles of therapy and revised recommendations MMWR Recomm Rep. 47(RR-20):1-58; and Centers for Disease Control and Prevention (2012) Latent Tuberculosis Infection: A Guide for Primary Health Care Providers available from: http://www.cdc.gov/tb/publications/ltbi/treatment.htm. Accessed Nov. 13, 2012; and for Buruli ulcer, standard of care include drug therapy, surgery, and anti-bacterial agents, see WHO (eds. 2012) Treatment of Mycobacterium ulcerans disease (Buruli ulcer): guidance for health workers WHO ISBN9789241503402, see http://www.who.int/buruli/treatment/en/ and http://apps.whoint/iris/bitstream/10665/77771/1/9789241503402_eng.pdf; Lehman, et al. (eds. 2006) Buruli Ulcer: Prevention of Disability (POD) WHO ISBN13:9789241546812, see http://whqlibdoc.who.int/hq/2008/WHO_HTM_NTD_IDM_GBUI_2008.1_eng.pdf; and Portaels (ed. 2014) Laboratory Diagnosis of Buruli Ulcer: A manual for health care providers WHO ISBN:9789241505703, see http://apps.who.int/iris/handle/10665/111738 and http://www.who.int/iris/bitstream/10665/111738/http://apps.who.int//iris/bitstream/10665/11173 8/1/9789241505703_eng.pdf.

### VIII. Administration

The route of administration and dosage will vary with the infecting bacteria strain(s), the site and extent of infection (e.g., local or systemic), and the subject being treated. The routes of administration include but are not limited to: oral, aerosol or other device for delivery to the lungs, nasal spray, intravenous (IV), intramuscular, intraperitoneal, intrathecal, intraocular, vaginal, rectal, topical, lumbar puncture, intrathecal, and direct application to the brain and/or meninges. Excipients which can be used as a vehicle for the delivery of the therapeutic will be apparent to those skilled in the art. For example, the biologic and/or chemotherapeutic could be in lyophilized form and be dissolved just prior to administration by IV injection. The dosage of administration is contemplated to be in the range of about 0.03, 0.1, 0.3, 1, 3, 10, 30, 100, 300, 1000, 3000, 1E4, 3×10E4, 10E5, 3×10E5, 10E6, 3×10E6, 10E7, 3×10E7 or more biologic molecules per bacterium in the host infection. Depending upon the size of the biologic, which may itself be tandemly associated, or in multiple subunit form (dimer, trimer, tetramer, pentamer, and the like) or in combination with one or more other entities, e.g., enzymes or fragments of different specificity, the dose may be about 1 million to about 10 trillion/per kg/per day, and preferably about 1 trillion/per kg/per day, and may be from about 10E6 linkage cleavage units/kg/day to about 10E13 linkage cleavage units/kg/day.

The chemotherapeutic component of the combination will generally be administered similarly to how it is used when not in combination with the biologic, though preferably in a smaller number of chemotherapeutic entities, at lower dosage, and/or for a shorter period of treatment. In certain circumstances, the combination may require modification of one or the other component compared to use alone, which may include modifications in timing, rate of administration, route of administration, or other aspect of dosing or administration.

Methods to evaluate mycobacteria killing capacity of the presently disclosed combinations are similar to methods used to evaluate therapeutic efficacy of standard mycobacteria therapies. Serial dilutions of bacterial cultures exposed to the compositions can quantify minimum dosages. Alternatively, comparing total bacterial counts with viable colony units can establish how many, or the fraction of mycobacteria are viable, and how many have been eliminated.

The therapeutic(s) are typically administered until successful elimination of the pathogenic mycobacteria is achieved, though broad spectrum formulations may be used while specific diagnosis of the infecting strain is being determined. Thus single dosage forms, as well as multiple dosage forms of the presently disclosed compositions are contemplated, as are methods for accomplishing sustained release means for delivery of such single and multi-dosages forms.

With respect to the aerosol administration to the lungs or other mucosal surfaces, the therapeutic composition is incorporated into an aerosol formulation specifically designed for administration. An example of such an aerosol is the Proventil inhaler manufactured by Schering-Plough, the propellant of which contains trichloromonofluoromethane, dichlorodifluoromethane, and oleic acid. Other embodiments include inhalers that are designed for administration to nasal and sinus passages of a subject or patient. The concentrations of the propellant ingredients and emulsifiers are adjusted if necessary based on the specific composition being used in the treatment. The number of outer membrane acting biologic molecules to be administered per aerosol treatment will typically be in the range of about 10E6 to 10E17 molecules, and preferably about 10E12.

Typically, the therapy will decrease bacterial replication capacity by at least about 3 fold, and may affect it by about 10, 30, 100, 300, etc., to many orders of magnitude. However, even slowing the rate of bacterial replication without killing may have significant therapeutic or commercial value. Genetic inactivation efficiencies are typically 0.1, 0.2, 0.3, 0.5, 0.8, 1, 1.5, 2.0, 2.5, 3.0, 3.5, 4, 5, 6, 7, 8, or more log units.

### IX. Formulations

The presently disclosed compositions and methods further include pharmaceutical compositions comprising at least one outer membrane acting biologic with the chemotherapeutic(s), e.g., an esterase, lipase, cutinase, or α/β hydrolase, provided in a pharmaceutically acceptable excipient. The formulations and pharmaceutical compositions thus include formulations comprising, with or without mycobacteria chemotherapeutic, an isolated biologic specific for a mycobacterium; a mixture of two, three, five, ten, or twenty or more biologics that affect the same or typical bacterial host; and a mixture of two, three, five, ten, or twenty or more biologics that affect different bacteria or different strains of the same bacterium, e.g., a cocktail mixture of biologics that collectively increase the permeability of the outer membrane of the target mycobacteria, of the macrophage, and/or the fibroblasts sequestering the granuloma. In this manner, the presently disclosed compositions of can be tailored to the needs of the patient. The compounds or compositions will typically be sterile or near sterile.

The term “therapeutically effective dose” indicates a dose of each component or combination that produces the effect(e.g., bacteriostatic or bactericidal) for which it is administered. The exact dose will depend on the purpose of the treatment, and will be ascertainable by one skilled in the art using known techniques. See, e.g., Ansel, et al. Pharmaceutical Dosage Forms and Drug Delivery; Lieberman (1992) Pharmaceutical Dosage Forms (vols. 1-3), Dekker, ISBN 0824770846, 082476918X, 0824712692, 0824716981; Lloyd (1999) The Art, Science and Technology of Pharmaceutical Compounding; and Pickar and Pickar-Abernethy (2012) Dosage Calculations, Delmar Cengage Learning, ISBN-10: 1439058474, ISBN013: 9781439058473. As is known in the art, adjustments for protein degradation, systemic versus localized delivery, and rate of new protease synthesis, as well as the age, body weight, general health, sex, diet, time of administration, drug interaction, spectrum of bacterial components in the colony, and the severity of the condition may be necessary, and will be ascertainable with some experimentation by those skilled in the art. In particular, relative amounts of the outer membrane acting biologic, other biologic or polypeptide, and chemotherapeutic may be adjusted and tested for optimal combinations. In particular, the combinations may increase the efficacy of various components such that other components may be reduced or eliminated from the combination. Alternatively, the combination may reduce effective treatment time, which allows for termination of the course of therapy after a shorter term.

Various pharmaceutically acceptable excipients are well known in the art. As used herein, “pharmaceutically acceptable excipient” includes a material which, when combined with an active ingredient of a composition, allows the ingredient to retain biological activity and without causing disruptive reactions with the subject's immune or other systems. Such may include stabilizers, preservatives, salt, or sugar complexes or crystals, solubilizing agents, and the like.

Exemplary pharmaceutically carriers include sterile aqueous of non-aqueous solutions, suspensions, and emulsions. Examples include, but are not limited to, standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In other embodiments, the compositions will be incorporated into solid matrix, including slow release particles, glass beads, bandages, inserts on the eye, and topical forms.

A composition comprising a biologic as described herein can also be lyophilized using means well known in the art, e.g., for subsequent reconstitution and use as disclosed.

Also of interest are formulations for liposomal delivery, and formulations comprising microencapsulated biologics, including sugar crystals. Compositions comprising such excipients are formulated by well-known conventional methods (see, e.g., Remington's Pharmaceutical Sciences, Chapter 43, 14th Ed., Mack Publishing Col, Easton Pa. 18042, USA).

Pharmaceutical compositions can be prepared in various forms, such as granules, tablets, pills, suppositories, capsules (e.g. adapted for oral delivery), microbeads, microspheres, liposomes, suspensions, salves, lotions and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Formulations may incorporate stabilizing agents, wetting and emulsifying agents, salts for varying the osmotic pressure or buffers for securing an adequate pH value.

The pharmaceutical composition can comprise other components in addition to the outer membrane acting biologic. In addition, the pharmaceutical compositions may comprise more than one active ingredient, e.g., two or more, three or more, five or more, or ten or more different biologics, where the different biologics may be specific for the same, different, or accompanying bacteria. For example, the pharmaceutical composition can contain multiple (e.g., at least two or more) defined outer membrane acting biologics, wherein at least two of the biologics in the composition have different mycobacteria specificity. In this manner, the therapeutic composition can be adapted for treating a mixed infection of different mycobacteria, or may be a composition selected to be effective against various types of infections found commonly in a particular institutional environment. A select combination may result, e.g., by selecting different groups of outer membrane acting entities derived from various sources of differing specificity so as to contain at least one component effective against different or critical bacteria (e.g., strain, species, etc.) suspected of being present in the infection (e.g., in the infected site) or typically accompanying such infection. As noted above, the outer membrane acting biologic can be administered in conjunction with other agents, such as biologics which affect macrophage permeability or granuloma permeability, or with one or more conventional anti-mycobacteria chemotherapeutic. In some embodiments, it may be desirable to administer the biologic and antibiotic within the same formulation. Alternatively, different therapeutics may be administered in succession.

### X. Methodology

In some embodiments, the presently disclosed compositions and methods involve well-known methods general clinical microbiology, general methods for handling bacteriophage, and general fundamentals of biotechnology principles and methods. References for such methods are listed below and are herein incorporated by reference for all purposes.

A. General Clinical Microbiology

General microbiology is the study of the microorganisms. See, e.g., Sonenshein, et al. (eds. 2002) Bacillus Subtilis and Its Closest Relatives: From Genes to Cells Amer. Soc. Microbiol., ISBN: 1555812058; Alexander and Strete (2001) Microbiology: A Photographic Atlas for the Laboratory Benjamin/Cummings, ISBN: 0805327320; Cann (2001) Principles of Molecular Virology (Book with CD-ROM; 3d ed.), ISBN: 0121585336; Garrity (ed. 2005) Bergey's Manual of Systematic Bacteriology (2 vol. 2d ed.) Plenum, ISBN: 0387950400; Salyers and Whitt (2001) Bacterial Pathogenesis: A Molecular Approach (2d ed.) Amer. Soc. Microbiol., ISBN: 155581171 X; Tiemo (2001) The Secret Life of Germs: Observations and Lessons from a Microbe Hunter Pocket Star, ISBN: 0743421876; Block (ed. 2000) Disinfection, Sterilization., and Preservation (5th ed.) Lippincott Williams & Wilkins Publ., ISBN: 0683307401; Cullimore (2000) Practical Atlas for Bacterial Identification Lewis Pub., ISBN: 1566703921; Madigan, et al. (2000) Brock Biology of Microorganisms (9th ed.) Prentice Hall, ASIN: 0130819220; Maier, et al. (eds. 2000) Environmental Microbiology Academic Pr., ISBN: 0124975704; Tortora, et al. (2000) Microbiology: An Introduction including Microbiology Place™ Website, Student Tutorial CD-ROM, and Bacteria ID CD-ROM (7th ed.), Benjamin/Cummings, ISBN 0805375546; Demain, et al. (eds. 1999) Manual of Industrial Microbiology and Biotechnology (2d ed.) Amer. Soc. Microbiol., ISBN: 1555811280; Flint, et al. (eds. 1999) Principles of Virology: Molecular Biology, Pathogenesis, and Control Amer. Soc. Microbiol., ISBN: 1555811272; Murray, et al. (ed. 1999) Manual of Clinical Microbiology (7th ed.) Amer. Soc. Microbiol., ISBN: 1555811264; Burlage, et al. (eds. 1998) Techniques in Microbial Ecology Oxford Univ. Pr., ISBN: 0195092236; Forbes, et al. (1998) Bailey & Scott's Diagnostic Microbiology (10th ed.) Mosby, ASIN: 0815125356; Schaechter, et al. (ed. 1998) Mechanisms of Microbial Disease (3d ed.) Lippincott, Williams & Wilkins, ISBN: 0683076051; Tomes (1998) The Gospel of Germs: Men, Women, and the Microbe in American Life Harvard Univ. Pr., ISBN: 0674357078; Snyder and Champness (1997) Molecular Genetics of Bacteria Amer. Soc. Microbiol., ISBN: 1555811027; Karlen (1996) Man and Microbes: Disease and Plagues in History and Modern Times Touchstone Books, ISBN: 0684822709; and Bergey (ed. 1994) Bergey's Manual of Determinative Bacteriology (9th ed.) Lippincott, Williams & Wilkins, ISBN: 0683006037.

B. General Methods for Handling Bacteriophage

General methods for handling bacteriophage are well known, see, e.g., Snustad and Dean (2002) Genetics Experiments with Bacterial Viruses Freeman; O'Brien and Aitken (eds. 2002) Antibody Phage Display: Methods and Protocols Humana; Ring and Blair (eds. 2000) Genetically Engineered Viruses BIOS Sci. Pub.; Adolf (ed. 1995) Methods in Molecular Genetics: Viral Gene Techniques vol. 6, Elsevier; Adolf (ed. 1995) Methods in Molecular Genetics: Viral Gene Techniques vol. 7, Elsevier; and Hoban and Rott (eds. 1988) Molec. Biol. of Bacterial Virus Systems (Current Topics in Microbiology and Immunology No. 136) Springer-Verlag.

C. General-Fundamentals of Biotechnology, Principles and Methods

General fundamentals of biotechnology, principles and methods are described, e.g., in Alberts, et al. (2002) Molecular Biology of the Cell (4th ed.) Garland ISBN: 0815332181; Lodish, et al. (1999) Molecular Cell Biology (4th ed.) Freeman, ISBN: 071673706X; Janeway; et al. (eds. 2001) Immunobiology (5th ed.) Garland, ISBN: 081533642X; Flint, et al. (eds. 1999) Principles of Virology: Molecular Biology, Pathogenesis, and Control, Am. Soc. Microbiol., ISBN: 1555811272; Nelson, et al. (2000) Lehninger Principles of Biochemistry (3d ed.) Worth, ISBN: 1572599316; Freshney (2000) Culture of Animal Cells: A Manual of Basic Technique (4th ed.) Wiley-Liss; ISBN: 0471348899; Arias and Stewart (2002) Molecular Principles of Animal Development, Oxford University Press, ISBN: 0198792840; Griffiths, et al. (2000) An Introduction to Genetic Analysis (7th ed.) Freeman, ISBN: 071673771X; Kierszenbaum (2001) Histology and Cell Biology, Mosby, ISBN: 0323016391; Weaver (2001) Molecular Biology (2d ed.) McGraw-Hill, ISBN: 0072345179; Barker (1998) At the Bench: A Laboratory Navigator CSH Laboratory, ISBN: 0879695234; Branden and Tooze (1999) Introduction to Protein Structure (2d ed.), Garland Publishing; ISBN: 0815323050; Sambrook and Russell (2001) Molecular Cloning: A Laboratory Manual (3 vol., 3d ed.), CSH Lab. Press, ISBN: 0879695773; Green and Sambrook (2012) Molecular Cloning: A Laboratory Manual (4th ed.) CSH Press, ISBN-10: 1605500569, ISBN-13: 978-1936113422; Ausubel (ed. 2002) Short Protocols in Molecular Biology (5th ed.), Wiley, ISBN-10: 0471250929, ISBN-13: 978-0471250920; Ausubel (ed. 1995) Current Protocols in Molecular Biology, Wiley & Sons, ISBN-10: 047150338X, ISBN-13: 978-0471503385; Ausubel (ed. 1987) Current Protocols in Molecular Biology, Wiley Online Library, ISBN-10: 0471625949, ISBN-13: 978-0471625940; and Scopes (1994) Protein Purification: Principles and Practice (3d ed.) Springer Verlag, ISBN: 0387940723.

D. Mutagenesis; Site Specific, Random, Shuffling

Based upon the structural and functional descriptions provide herein, homologs and variants may be isolated or generated which may optimize preferred features. Thus, additional catalytic segments of permeability functions may be found by structural homology, or by evaluating entities found in characteristic gene organization motifs. Microbiologic or eukaryotic genes may be identified by gene arrangement characteristic of genes having function, and may be found in particular gene arrangements, and other entities found in the corresponding arrangements can be tested for a mycobacteria outer membrane permeabilizing function. These may also serve as the starting points to screen for variants of the structures, e.g., mutagenizing such structures and screening for those which have desired characteristics, e.g., broader substrate specificity. Standard methods of mutagenesis may be used, see, e.g., Johnson-Boaz, et al. (1994) Mol. Microbiol. 13:495-504; U.S. Pat. Nos. 6,506,602, 6,518,065, 6,521,453, 6,579,678, and references cited by or therein.

Binding or targeting segments can be attached (e.g., in a fusion protein) to the presently described biologics. Prevalent or specific target motifs can be screened for binding domains which interact specifically with them. The target can be a highly expressed protein, carbohydrate, or lipid containing structures found on a particular target strains. While many proteins are known to bind to mycobacteria cell walls, attractive options include, e.g., BD domain (e.g., from LysA, LysB from D29, or other mycobacteriophages) (Pohane et al. (2014) “Molecular dissection of phage endolysin: An interdomain interaction confers specificity in Lysin A of Mycobacterium host phage D29” J. Biol. Chem. 289:12085-12095); LysM of Bacillus phage (Morita, et al. (2001) “Functional analysis of antibacterial activity of Bacillus amyloliquefaciens phage endolysin against Gram-negative bacteria” FEBS Letters 200:56-59).

The components of the mycobacteria cell wall may be shared with components of other bacteria cell walls, or possibly with other mycobacteria or spores. Phage or bacteria sharing structural features are sources to find functions which can degrade such linkages.

A targeting moiety may increase a local concentration of a catalytic fragment, but a linker of appropriate length may also increase the number of mycobacteria outer membrane cleavage events locally. Thus, linkers compatible with the target and catalytic motifs or of appropriate length may be useful and increase the permeability enhancing activity leading greater accessibility of the chemotherapeutics, which may contribute to stasis or killing of target mycobacteria.

E. Screening

Screening methods can be devised for evaluating mutants or new candidate functional segments. A library of different outer membrane acting biologics could be screened for presence of such gene products. Binding may use crude bacteria cultures, isolated mycobacteria cell wall components, peptidoglycan preparations, synthetic substrates, or purified reagents to determine the affinity and number of interactions on target cells. Permeability or wall degrading assays may be devised to evaluate integrity of the mycobacteria outer membrane of target strains, lawn inhibition assays, viability tests of cultures, activity on cell wall preparations or other substrates, or release of components (e.g., sugars, amino acids, polymers) of the cell wall or mycobacteria outer membrane upon catalytic action.

Linker features may be tested to compare the effects on binding or catalysis of particular linkers, or to compare the various orientations of fragments. Panels of targets may be screened for catalytic fragments which act on a broader or narrower spectrum of target mycobacteria, and may include other microbes which may share cell wall components, e.g., spores. This may make use of broader panels of related mycobacteria strains. Strategies may be devised which allow for screening of larger numbers of candidates or variants.

One method to test for a permeabilizing or cell wall degrading activity is to treat source microorganisms with mild detergents to release structurally associated proteins. These proteins are further tested for permeabilizing or wall degrading activity on mycobacteria cells. The permeability assays may evaluate permeability from outside the cell to in, or inside to out.

### XI. Nucleic Acids Encoding Mycobacteria Outer Membrane Acting Biologics

Nucleic acids have been identified that encode the outer membrane or cell wall acting biologics described above, e.g., phage or bacterial LysB-like biologics. Encoded mycobacteria outer membrane acting proteins may have outer membrane degrading activity, and those encoding identified Pfam domains are prime candidates, especially those in the listed Pfams. Alternative sources include genomic sequences which possess characteristic features of “lytic” activity containing elements.

Nucleic acids that encode mycobacteria outer membrane or cell wall acting biologics are included in the presently disclosed compositions and methods. Methods of obtaining such nucleic acids will be recognized by those of skill in the art. Suitable nucleic acids (e.g., cDNA, genomic, or subsequences (probes)) can be cloned, or amplified by in vitro methods such as the polymerase chain reaction (PCR), the ligase chain reaction (LCR), the transcription-based amplification system (TAS), or the self-sustained sequence replication system (SSR). Besides synthetic methodologies, a wide variety of cloning and in vitro amplification methodologies are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook, et al. (1989) Molecular Cloning—A Laboratory Manual (2nd ed.) Vol. 1-3, Cold Spring Harbor Laboratory, Cold Spring Harbor Press, NY, (Sambrook, et al.); Current Protocols in Molecular Biology, Ausubel, et al., eds., Current Protocols, a joint venture between Greene Publishing Associates, Inc. and John Wiley & Sons, Inc., (1994 Supplement) (Ausubel); Cashion, et al., U.S. Pat. No. 5,017,478; and Carr, European Patent No. 0,246,864.

A DNA that encodes a mycobacteria outer membrane or cell wall acting biologic, can be prepared by a suitable method described above, including, e.g., cloning and restriction of appropriate sequences with restriction enzymes. In one preferred embodiment, nucleic acids encoding mycobacteria outer membrane permeabilizing polypeptides are isolated by routine cloning methods. A nucleotide sequence of a mycobacteria outer membrane or cell wall acting biologic as provided, e.g., in LysB Accession Number NP_046827.1 or NC_001900.1 can be used to provide probes that specifically hybridize to a gene encoding the polypeptide; or to an mRNA, encoding a mycobacteria outer membrane permeabilizing biologic, in a total RNA sample (e.g., in a Southern or Northern blot). Once the target nucleic acid encoding a mycobacteria outer membrane or cell wall acting biologic is identified, it can be isolated according to standard methods known to those of skill in the art (see, e.g., Sambrook, et al. (1989) Molecular Cloning: A Laboratory Manual (2d ed., Vols. 1-3) Cold Spring Harbor Laboratory; Berger and. Kimmel (1987) Methods in Enzymology, Vol. 152: Guide to Molecular Cloning Techniques, San Diego: Academic Press, Inc.; or Ausubel, et al. (1987) Current Protocols in Molecular Biology, Greene Publishing and Wiley-Interscience, New York). Further, the isolated nucleic acids can be cleaved with restriction enzymes to create nucleic acids encoding the full-length mycobacteria outer membrane permeabilizing polypeptide, or subsequences thereof, e.g., containing subsequences encoding at least a subsequence of a catalytic domain of a mycobacteria outer membrane permeabilizing polypeptide. These restriction enzyme fragments, encoding a mycobacteria outer membrane permeabilizing polypeptide or subsequences thereof, may then be ligated, for example, to produce a nucleic acid encoding a mycobacteria outer membrane permeabilizing polypeptide.

Similar methods can be used to generate appropriate outer mycobacteria membrane binding fragments or linkers between fragments. Binding segments with affinity to prevalent surface features on target bacteria can be identified and include those from, e.g., LysB. Linker segments of appropriate lengths and properties can be used to connect binding and catalytic domains. See, e.g., Bae, et al. (2005) “Prediction of protein interdomain linker regions by a hidden Markov model” Bioinformatics 21:2264-2270; and George and Heringa (2003) “An analysis of protein domain linkers: their classification and role in protein folding” Protein Engineering 15:871-879.

A nucleic acid encoding an appropriate biologic, or a subsequence thereof, can be characterized by assaying for the expressed product. Assays based on the detection of the physical, chemical, or immunological properties of the expressed polypeptide can be used. For example, one can identify a mycobacteria outer membrane or cell wall acting polypeptide by the ability of a polypeptide encoded by the nucleic acid to increase permeability of mycobacteria, to degrade, or to digest mycobacteria cells, e.g., as described herein.

Also, a nucleic acid encoding a desired biologic, or a subsequence thereof, can be chemically synthesized. Suitable methods include the phosphotriester method of Narang, et al. (1979) Meth. Enzymol. 68: 90-99; the phosphodiester method of Brown, et al. (1979) Meth. Enzymol. 68:109-151; the diethylphosphoramidite method of Beaucage, et al. (1981) Tetra. Lett. 22:1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill recognizes that while chemical synthesis of DNA is often limited to sequences of about 100 bases, longer sequences may be obtained by the ligation of shorter sequences.

Nucleic acids encoding a desired polypeptide, or subsequences thereof, can be cloned using DNA amplification methods such as polymerase chain reaction (PCR). Thus, for example, the nucleic acid sequence or subsequence is PCR amplified, using a sense primer containing one restriction enzyme site (e.g., NdeI) and an antisense primer containing another restriction enzyme site (e.g., HindIII). This will produce a nucleic acid encoding the desired polypeptide or subsequence and having terminal restriction enzyme sites. This nucleic acid can then be easily ligated into a vector containing a nucleic acid encoding the second molecule and having the appropriate corresponding restriction enzyme sites. Suitable PCR primers can be determined by one of skill in the art using sequence information provided, e.g., in GenBank or other sources. Appropriate restriction enzyme sites can also be added to the nucleic acid encoding the mycobacteria outer membrane permeabilizing biologic or polypeptide subsequence thereof by site-directed mutagenesis. The plasmid containing a mycobacteria outer membrane permeabilizing biologic-encoding nucleotide sequence or subsequence is cleaved with the appropriate restriction endonuclease and then ligated into an appropriate vector for amplification and/or expression according to standard methods. Examples of techniques sufficient to direct persons of skill through in vitro amplification methods are found in Berger, Sambrook, and Ausubel, as well as Mullis, et al. (1987) U.S. Pat. No. 4,683,202; PCR Protocols A Guide to Methods and Applications (Innis, et al., eds.) Academic Press Inc. San Diego, Calif. (1990) (Innis); Arnheim and Levinson (Oct. 1, 1990) C&EN36-47; The Journal Of NIH Research (1991) 3:81-94; (Kwoh, et al. (1989) Proc. Nat'l Acad. Sci. USA 86:1173; Guatelli, et al. (1990) Proc. Nat'l Acad. Sci. USA 87:1874; Lomeli, et al. (1989) J. Clin. Chem. 35:1826; Landegren, et al. (1988) Science 241:1077-1080; Van Brunt (1990) Biotechnology 8: 291-294; Wu and Wallace (1989) Gene 4:560; and Barringer, et al. (1990) Gene 89:117.

Some nucleic acids encoding mycobacteria outer membrane acting biologics can be amplified using PCR primers based on the sequence of the identified polypeptides.

Other physical properties, e.g., of a recombinant mycobacteria outer membrane acting biologic expressed from a particular nucleic acid, can be compared to properties of known desired polypeptides to provide another method of identifying suitable sequences or domains, e.g., of the outer membrane acting biologics that are determinants of bacterial specificity, binding specificity, and/or catalytic activity. Alternatively, a putative mycobacteria outer membrane acting biologic encoding nucleic acid or recombinant mycobacteria outer membrane permeabilizing biologic gene can be mutated, and its role as a permeabilizing biologic, or the role of particular sequences or domains established by detecting a variation in mycobacteria effect normally enhanced by the unmutated, naturally-occurring, or control mycobacteria outer membrane acting biologic. Mutation or modification of the presently disclosed polypeptides can be facilitated by molecular biology techniques to manipulate the nucleic acids encoding the polypeptides, e.g., PCR. Other mutagenesis or gene shuffling techniques can be applied to the functional fragments described herein, including mycobacteria outer membrane acting activities, cell wall acting properties, or linker features compatible with chimeric constructs.

Functional domains of newly identified mycobacteria outer membrane acting biologics can be identified by using standard methods for mutating or modifying the polypeptides and testing them for activities such as acceptor substrate activity and/or catalytic activity, as described herein. The sequences of functional domains of the various cell wall acting proteins can be used to construct nucleic acids encoding or combining functional domains of one or more cell wall acting polypeptides. These multiple activity polypeptide fusions can then be tested for a desired bactericidal or bacteriostatic activity. Related sequences based on homology to identified “lytic” activities can be identified and screened for activity on appropriate substrates.

In an exemplary approach to cloning nucleic acids encoding mycobacteria outer membrane acting polypeptides, the known nucleic acid or amino acid sequences of cloned polypeptides are aligned and compared to determine the amount of sequence identity between them. This information can be used to identify and select polypeptide domains that confer or modulate cell wall acting polypeptide activities, e.g., target bacterial or binding specificity and/or permeabilizing activity based on the amount of sequence identity between the polypeptides of interest. For example, domains having sequence identity between the outer membrane acting polypeptides of interest, and that are associated with a known activity, can be used to construct polypeptides containing that domain and other domains, and having the activity associated with that domain (e.g., bacterial or binding specificity and/or outer membrane permeabilizing activity).

### XII. Expression of Desired Biologics in Host Cells

Mycobacteria outer membrane acting (or other) biologics of can be expressed in a variety of host cells, including E. coli, other bacterial hosts, and yeast. The host cells are preferably microorganisms, such as, e.g., yeast cells, bacterial cells, or filamentous fungal cells. Examples of suitable host cells include, for example, Azotobacter sp. (e.g., A. vinelandii), Pseudomonas sp., Rhizobium sp., Erwinia sp., Escherichia sp. (e.g., E. coli), Bacillus, Pseudomonas, Proteus, Salmonella, Serratia, Shigella, Rhizobia, Vitreoscilla, Paracoccus and Klebsiella sp., among many others. The cells can be of any of several genera, including Saccharomyces (e.g., S. cerevisiae), Candida (e.g., C. utilis, C. parapsilosis, C. krusei, C. versatilis, C. lipolytica, C. zeylanoides, C. guilliermondii, C. albicans, and C. humicola), Pichia (e.g., P. farinosa and P. ohmeri), Torulopsis (e.g., T. candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailii), Kluyveromyces (e.g., K. marxianus), Hansenula (e.g., H. anomala and H. jadinii), and Brettanomyces (e.g., B. lambicus and B. anomalus). Examples of useful bacteria include, but are not limited to, Escherichia, Enterobacter, Azotobacter, Erwinia, Klebsielia, Bacillus, Pseudomonas, Proteus, and Salmonella.

Once expressed in a host cell, the mycobacteria outer membrane acting biologics can be used to prevent growth of appropriate bacteria, typically in combination with the chemotherapeutics. In some embodiments, a LysB biologic is used to decrease growth of a mycobacterium. In some embodiments, the protein is used to decrease growth, or affect mycobacteria outer membrane permeability, of a tuberculosis bacterium, or other similar mycobacteria species. Fusion constructs combining such fragments can be generated, including fusion proteins comprising a plurality of mycobacteria outer membrane or cell wall permeabilizing activities, including both peptidase and esterase catalytic activities, or combining the activity with another segment, e.g., a targeting segment which binds to cell wall structures, or a permeabilizing segment which provides macrophage or granuloma permeability. Combinations of degrading activities can act synergistically for better bacteriostatic or bactericidal activity by an accompanying chemotherapeutic. A linker can be incorporated to provide additional volume for catalytic sites of high local concentration near the binding target.

Typically, a polynucleotide that encodes the mycobacteria outer membrane acting biologics is placed under the control of a promoter that is functional in the desired host cell. An extremely wide variety of promoters is well known, and can be used in expression vectors, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, provided herein are expression cassettes into which the nucleic acids that encode fusion proteins, e.g., combining a catalytic fragment with a binding fragment, are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change, et al. (1977) Nature 198:1056), the tryptophan (trp) promoter system (Goeddel, et al. (1980) Nucleic Acids Res. 8:4057), the tac promoter (DeBoer, et al. (1983) Proc. Nat'l Acad. Sci. USA 80:21-25); and the lambda-derived P.sub.L promoter and N-gene ribosome binding site (Shimatake, et al. (1981) Nature 292:128). The particular promoter system is typically not critical; many available promoters that function in prokaryotes can be used. A bacteriophage T7 promoter is used as an example.

For expression of outer membrane acting polypeptides in prokaryotic cells other than E. coli, a promoter that functions in the particular prokaryotic production species is used. Such promoters can be obtained from genes that have been cloned from the species, or heterologous promoters can be used. For example, the hybrid trp-lac promoter functions in Bacillus in addition to E. coli.

A ribosome binding site (RBS) is conveniently included in an expression cassette. An exemplary RBS in E. coli consists of a nucleotide sequence 3-9 nucleotides in length located 3-11 nucleotides upstream of the initiation codon (Shine and Dalgarno (1975) Nature 254:34; Steitz in Goldberger (ed. 1979) Biological regulation and development: Gene expression (vol. 1, p. 349) Plenum Publishing, NY).

For expression of proteins in yeast, convenient promoters include GAL1-10 (Johnson and Davies (1984) Mol. Cell. Biol. 4:1440-1448) ADH2 (Russell, et al. (1983) J. Biol. Chem. 258:2674-2682), PHOS (EMBO J. (1982) 6:675-680), and MF.alpha. (Herskowitz and Oshima (1982) in Strathern, et al. (eds.) The Molecular Biology of the Yeast Saccharomyces Cold Spring Harbor Lab., Cold Spring Harbor, N.Y., pp. 181-209). Another suitable promoter for use in yeast is the ADH2/GAPDH hybrid promoter as described in Cousens, et al. (1987) Gene 61:265-275 (1987). For filamentous fungi such as, for example, strains of the fungi Aspergillus (McKnight, et al., U.S. Pat. No. 4,935,349), examples of useful promoters include those derived from Aspergillus nidulans glycolytic genes, such as the ADH3 promoter (McKnight, et al. (1985) EMBO J. 4:2093-2099) and the tpiA promoter. An example of a suitable terminator is the ADH3 terminator (McKnight, et al.).

Either constitutive or regulated promoters can be used. Regulated promoters can be advantageous because the host cells can be grown to high densities before expression of the fusion proteins is induced. High level expression of heterologous polypeptides slows cell growth in some situations. An inducible promoter is a promoter that directs expression of a gene where the level of expression is alterable by environmental or developmental factors such as, for example, temperature, pH, anaerobic or aerobic conditions, light, transcription factors, and chemicals. Such promoters are referred to herein as “inducible” promoters, which allow one to control the timing of expression of the desired polypeptide. For E. coli and other bacterial host cells, inducible promoters are known to those of skill in the art. These include, for example, the lac promoter, the bacteriophage lambda PL promoter, the hybrid trp-lac promoter (Amann, et al. (1983) Gene 25:167; de Boer, et al. (1983) Proc. Nat'l Acad. Sci. USA 80:21), and the bacteriophage T7 promoter (Studier, et al. (1986) J. Mol. Biol.; Tabor, et al. (1985) Proc. Nat'l Acad. Sci. USA 82:1074-78). These promoters and their use are discussed in Sambrook, et al., supra.

A construct that includes a polynucleotide of interest (e.g., outer membrane acting biologic) operably linked to gene expression control signals that, when placed in an appropriate host cell, drive expression of the polynucleotide is termed an “expression cassette.” Expression cassettes that encode fusion proteins are often placed in expression vectors for introduction into the host cell. The vectors typically include, in addition to an expression cassette, a nucleic acid sequence that enables the vector to replicate independently in one or more selected host cells. Generally, this sequence is one that enables the vector to replicate independently of the host chromosomal DNA, and includes origins of replication or autonomously replicating sequences. Such sequences are well known for a variety of bacteria. For instance, the origin of replication from the plasmid pBR322 is suitable for most Gram-negative bacteria. Alternatively, the vector can replicate by becoming integrated into the host cell genomic complement and being replicated as the cell undergoes DNA replication.

The construction of polynucleotide constructs generally requires the use of vectors able to replicate in bacteria. A plethora of kits are commercially available for the purification of plasmids from bacteria (see, e.g., EasyPrepJ, FlexiPrepJ, both from Pharmacia Biotech; StrataClean, from Stratagene; and, QlAexpress Expression System, Qiagen). The isolated and purified plasmids can then be further manipulated to produce other plasmids, and used to transfect cells. Cloning in Streptomyces or Bacillus is also possible.

Selectable markers are often incorporated into the expression vectors used to express the desired polynucleotides. These genes can encode a gene product, such as a polypeptide, necessary for the survival or growth of transformed host cells grown in a selective culture medium. Host cells not transformed with the vector containing the selection gene will not survive in the culture medium. Typical selection genes encode polypeptides that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the host cell. A number of selectable markers are known to those of skill in the art and are described for instance in Sambrook, et al., supra.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques Methods in Enzymology, Volume 152, Academic Press, Inc., San Diego, Calif. (Berger); and Current Protocols in Molecular Biology, Ausubel, et al., eds., Current Protocols, a joint venture between Greene Publishing Associates, Inc. and John Wiley & Sons, Inc. (1998 Supplement) (Ausubel).

A variety of common vectors suitable for use as starting materials for constructing the presently disclosed expression vectors are well known in the art. For cloning in bacteria, common vectors include pBR322 derived vectors such as pBLUESCRIPTTM, and .lamda.-phage derived vectors. In yeast, vectors include Yeast Integrating plasmids (e.g., YIp5) and Yeast Replicating plasmids (the YRp series plasmids) and pGPD-2. Expression in mammalian cells can be achieved using a variety of commonly available plasmids, including pSV2, pBC12BI, and p91023, as well as lytic virus vectors (e.g., vaccinia virus, adenovirus, and baculovirus), episomal virus vectors (e.g., bovine papillomavirus), and retroviral vectors (e.g., murine retroviruses).

The methods for introducing the expression vectors into a chosen host cell are typically standard, and such methods are known to those of skill in the art. For example, the expression vectors can be introduced into prokaryotic cells, including E. coli, by calcium chloride transformation, and into eukaryotic cells by calcium phosphate treatment or electroporation. Other transformation methods are also suitable.

Translational coupling can be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et al. (1988) J. Biol. Chem. 263: 16297-16302.

The polypeptides disclosed herein can be expressed intracellularly, or can be secreted from the cell. Intracellular expression often results in high yields. If necessary, the amount of soluble, active fusion polypeptide can be increased by performing refolding procedures (see, e.g., Sambrook, et al., supra; Marston, et al. (1984) Bio/Technology 2:800; Schoner, et al. (1985) Bio/Technology 3:151). In embodiments in which the desired polypeptide are secreted from the cell, either into the periplasm or into the extracellular medium, the DNA sequence is often linked to a cleavable signal peptide sequence. The signal sequence directs translocation of the fusion polypeptide through the cell membrane. An example of a suitable vector for use in E. coli that contains a promoter-signal sequence unit is pTA1529, which has the E. coli phoA promoter and signal sequence (see, e.g., Sambrook, et al., supra; Oka, et al. (1985) Proc. Nat'l Acad. Sci. USA 82:7212; Talmadge, et al. (1980) Proc. Nat'l Acad. Sci. USA 77:3988; Takahara, et al. (1985) J. Biol. Chem. 260:2670). In another embodiment, the fusion polypeptides are fused to a subsequence of protein A or bovine serum albumin (BSA), for example, to facilitate purification, secretion, or stability. Affinity methods, e.g., using the target of the binding fragment can be used.

The mycobacteria outer membrane permeabilizing biologics described herein can also be further linked to other bacterial polypeptide segments, e.g., targeting fragments or permeability segments. This approach often results in high yields, because normal prokaryotic control sequences direct transcription and translation. In E. coli, lacZ fusions are often used to express heterologous proteins. Suitable vectors are readily available, such as the pUR, pEX, and pMR100 series (see, e.g., Sambrook, et al., supra). For certain applications, extraneous sequence can be cleaved from the fusion polypeptide after purification. This can be accomplished by any of several methods known in the art, including cleavage by cyanogen bromide, a protease, or by Factor X.sub.a (see, e.g., Sambrook, et al., supra; Itakura, et al. (1977) Science 198:1056; Goeddel, et al. (1979) Proc. Nat'l Acad. Sci. USA 76:106; Nagai, et al. (1984) Nature 309:810; Sung, et al. (1986) Proc. Nat'l Acad. Sci. USA 83:561). Cleavage sites can be engineered into the gene for the fusion polypeptide at the desired point of cleavage.

More than one recombinant polypeptide can be expressed in a single host cell by placing multiple transcriptional cassettes in a single expression vector, or by utilizing different selectable markers for each of the expression vectors which are employed in the cloning strategy.

A suitable system for obtaining recombinant proteins from E. coli which maintains the integrity of their N-termini has been described by Miller, et al. (1989) Biotechnology 7:698-704. In this system, the gene of interest is produced as a C-terminal fusion to the first 76 residues of the yeast ubiquitin gene containing a peptidase cleavage site. Cleavage at the junction of the two moieties results in production of a protein having an intact authentic N-terminal reside.

### XIII. Purification of Desired Polypeptides

The presently disclosed polypeptides can be expressed as intracellular proteins or as proteins that are secreted from the cell. For example, a crude cellular extract containing the expressed intracellular or secreted polypeptides can be used in the presently disclosed methods.

Alternatively, the polypeptides can be purified according to standard procedures of the art, including ammonium sulfate precipitation, affinity columns, column chromatography, gel electrophoresis and the like (see, generally, Scopes (1982) Protein Purification Springer-Verlag, N.Y.; Deutscher (1990) Methods in Enzymology (vol. 182) Guide to Protein Purification, Academic Press, Inc. NY). Substantially pure compositions of at least about 70, 75, 80, 85, 90% homogeneity are preferred, and about 92, 95, 98 to 99% or more homogeneity are most preferred. The purified polypeptides can also be used, e.g., as immunogens for antibody production, which antibodies can be used in immunoselection purification methods.

To facilitate purification of polypeptides, the nucleic acids that encode them can also include a coding sequence for an epitope or “tag” for which an affinity binding reagent is available, e.g., a purification tag. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the presently disclosed polypeptides, and corresponding detection systems are known to those of skill in the art, and several are commercially available (e.g., FLAG, Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli “Purification of recombinant proteins with metal chelating adsorbents” in Setlow (ed. 1990) Genetic Engineering: Principles and Methods, Plenum Press, NY; commercially available from Qiagen (Santa Clarita, Calif.)). Purification tags also include maltose binding domains and starch binding domains. Purification of maltose binding domain proteins is known to those of skill in the art.

Other haptens that are suitable for use as tags are known to those of skill in the art and are described, for example, in the Handbook of Fluorescent Probes and Research Chemicals (6th ed., Molecular Probes, Inc., Eugene Oreg.). For example, dinitrophenol (DNP), digoxigenin, barbiturates (see, e.g., U.S. Pat. No. 5,414,085), and several types of fluorophores are useful as haptens, as are derivatives of these compounds. Kits are commercially available for linking haptens and other moieties to proteins and other molecules. For example, where the hapten includes a thiol, a heterobifunctional linker such as SMCC can be used to attach the tag to lysine residues present on the capture reagent.

One of skill would recognize that certain modifications can be made to the catalytic or functional domains of the polypeptide without diminishing their biological activity. Some modifications can be made to facilitate the cloning, expression, or incorporation of the catalytic domain into a fusion polypeptide. Such modifications are well known to those of skill in the art and include, for example, the addition of codons at either terminus of the polynucleotide that encodes the catalytic domain, e.g., a methionine added at the amino terminus to provide an initiation site, or additional amino acids (e.g., poly His) placed on either terminus to create conveniently located restriction enzyme sites or termination codons or purification sequences.

It must be noted that as used herein and in the appended claims, the singular forms “a”, “and”, and “the” include plural referents unless the context clearly dictates otherwise. Thus, e.g., reference to “a bacteriophage” includes a plurality of such bacteriophage and reference to a “host bacterium” includes reference to one or more host bacteria and equivalents thereof known to those skilled in the art, and so forth.

Publications discussed herein are provided solely for their disclosure prior to the filing date of the present application. All publications, websites, accession numbers, and patent literature cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the present disclosure that certain changes and modifications can be made thereto without departing from the spirit or scope of the appended claims.

### EXPERIMENTAL

### I. Identification of an OM Acting Enzymatic Activity; D29 LysB; Criteria for Selecting D29 LysB

The outer membrane of mycobacteria is a strong permeability barrier, which can isolate the cell from the exterior surroundings. The mycobacteria outer membrane contains a substantial amount of mycolic acids. As a means to target the outer membrane permeability barrier, enzymatic means were pursued to disrupt outer membrane function by detaching mycolic acid from the peptidoglycan. A phage encoded enzyme derived from a mycobacteria phage was identified for study. The removal of mycolic acids from these layers was hypothesized to increase the mycobacteria cell permeability and to have a direct effect on cell viability at higher protein concentrationby disintegrating the cell wall.

Mycobacteria Ms6 and D29 phages encode enzymes (LysB) with mAGP hydrolase activity. Gil, et al. (2010) “Mycobacteriophage Ms6 LysB, specifically targets the outer membrane of Mycobacterium smegmatis” Microbiology 156:1497-1504; and Payne, et al. (2009) “Mycobacteriophage Lysin B is a novel mycolylarabinogalactan Esterase” Mol. Microbiol. 73:367-381. D29 LysB was selected because it is highly bacteriocidal compared to other phages (Hassan, et al. (2010) “Lytic Efficiency of Mycobacteriophages” The Open Systems Biology Journal 3:21-28). In addition, D29 LysB protein is smaller in size compared to Ms6 phage LysB.

D29 LysB is one example, but other enzymes can be used, e.g., lysin B, Mycobacterium phage Chy5, accn no YP_008058282; Lysin B Mycobacterium phage L5, accn no NP_039676; gp14 Mycobacterium phage Trixie, accn no AEL17844.1; serine esterase, cutinase M. Smegmatis accn no YP_890104. Additional enzymes with mycobacteria outer membrane degrading activity include other activities such as lipase, cutinase, and α/β hydrolase enzymes as disclosed above.

### II. Purification Methods Used to Isolate the LysB

Recombinant D29 LysB was expressed as a His tagged protein in E. coli. The protein was purified through Ni affinity chromatography as described in detail below. Other affinity tags (e.g., GST, MBP, protein A, etc.) could also have been generated and used for affinity purification of the protein. Scopes (1994) Protein Purification: Principles and Practice (3d ed.) Springer Verlag, ISBN-10: 0387940723, ISBN-13: 978-0387940724; The QIAexpressionist (2001) A handbook for high-level expression and purification of 6×His-tagged proteins, QIAGEN.

The LysB gene (GeneID:1261627/D29_12) was amplified from mycobacteria phage D29 genomic DNA by PCR using appropriate primers (designated GMB763 and GMB764). The PCR conditions were as follows: Initial denaturation for 5 min at 95° C., followed by 29 cycles of denaturation at 95° C. for 30 sec, annealing at 55° C. for 30 sec., and extension at 72° C. for 45 sec respectively followed by the final extension of 72° C. for 30 sec. The PCR product was cloned into NdeI/HindIII sites of the pET26b plasmid (Novagen) to obtain pGDC403. The terminally 6X-His tagged protein was expressed in E. coli ER2566 as a soluble protein. The recombinant protein was purified by Ni-affinity chromatography and dialyzed against 25 mM Tris (pH 7.5). The dialyzed protein was stored at 4° C. till further use.

In variations, a protease cleavage site can be inserted in the expression construct to remove the His tag. The recombinant LysB (or other enzyme) can also be expressed and purified by conventional chromatography procedures, e.g., ammonium sulfate precipitation, ion exchange chromatography, and size exclusion chromatography. The protein is typically purified in a manner which preserves biological activity, but the protein can be renatured if necessary. See, e.g., Scopes (1994) Protein Purification: Principles and Practice (3d ed.) Springer Verlag, ISBN-10: 0387940723, ISBN-13: 978-0387940724; and Strategies for Protein Purification, GE Healthcare Life Sciences Handbook.

### III. Construction of Truncations, Mutagenesis for Functional Testing

Constructs for expressing N- and C-terminal deletions (e.g., in 10 AA incremental deletions) in LysB were generated by PCR by following standard protocols. See, e.g., Innes, et al (1990) PCR Protocols: A Guide to Methods and Applications Academic Press, ISBN-10: 0123721814, ISBN-13: 978-0123721815; Ausubel (ed. 2002) Short Protocols in Molecular Biology (5th ed.), Wiley, ISBN-10: 0471250929, ISBN-13: 978-0471250920; Ausubel (ed. 1995) Current Protocols in Molecular Biology, Wiley & Sons, ISBN-10: 047150338X, ISBN-13: 978-0471503385; Ausubel (ed. 1987) Current Protocols in Molecular Biology, Wiley Online Library, ISBN-10: 0471625949, ISBN-13: 978-0471625940; Green and Sambrook (2012) Molecular Cloning: A Laboratory Manual (4th ed.) CSH Press, ISBN-10: 1605500569, ISBN-13: 978-1936113422; and Sambrook, et al (2001) Molecular Cloning: A Laboratory Manual (3d ed. vol I, II and III) Cold Spring Harbor Laboratory Press, ISBN-10: 0879695773|ISBN-13: 978-0879695774. The results of activity testing indicated that less than the complete intact sequence can be used (e.g., 75% of the intact protein).

Mutagenesis studies can also be performed to find out what positions are critical for maintaining activity, and what positions of the polypeptide are tolerant of changes, which can include conservative or nonconservative amino acid substitutions, insertions, deletions, and chemical conjugations. The positions can be determined to enhance, decrease, or not affect activity, and the effect on activity of various different changes evaluated.

For example, a proposed active site Ser residue (Ser82) was mutated to Ala by site directed mutagenesis kit (Stratagene). Such substitution caused loss of esterase activity as well as loss of bactericidal property.

Alternatively, the truncated or mutant lysB gene can be synthesized by in vitro synthesis. The construct can incorporate various modifications or changes, e.g., purification tags, etc. Many commercial entities can be contracted to perform such, e.g., Genscript, Piscataway, N.J., USA; GeneArt Life Technologies, Bangalore, India or Grand Island, N.Y., USA; SinoBiological, Beijing, PRC; GeneWiz, Langen, Germany; and many others. The gene can then be expressed and tested for activity.

### IV. Combination Testing In Vitro and In Vivo

A. Detection of Synergy In Vitro

Synergy between LysB and anti-TB chemotherapeutic drugs (Rifampicin, Isoniazid and Ethambutol) was tested by checkerboard based MIC method. See, e.g., Franzblau, et al. (1998) “Rapid, low-technology MIC determination with clinical Mycobacterium tuberculosis isolates by using the microplate Alamar Blue assay” J. Clin. Microbiol. 36:362-6; and Solapure, et al. (2013) “In Vitro and In Vivo Efficacy of β-Lactams against Replicating and Slowly Growing/Nonreplicating Mycobacterium tuberculosis” Antimicrob. Agents Chemother. 57:2506-2510.

Decreasing concentrations of the drugs were used in rows of a microtiter plate, while decreasing concentrations of LysB were added to the columns of the plate. The mycobacteria cells were exposed to the drug combination for a specified duration of time and growth/no growth was detected by addition of 0.02% Resazurin to the wells of the plate. The plates were read spectrophotometrically at 2 different wavelengths, T1-575 nM and T2-610 nM, T1/T2 and reduced value was recorded. Media control was considered as 0% growth and culture control as 100% growth. The least concentration of drug giving, e.g., 40%, or 80% inhibition of growth was considered as the minimal inhibitory concentration (MIC) well. The synergy between LysB and anti-TB drugs was determined by calculating the fractional inhibitory concentration (FIC) index. See Franzblau, et al. (1998) J. Clin. Microbiol. 36:362-6; Ramon-Garcia, et al (2011) “Synergistic Drug Combinations for Tuberculosis Therapy Identified by a Novel High-Throughput Screen” Antimicrob. Agents Chemother. 55:3861-3869; and Solapure, et al. (2013) Antimicrob. Agents Chemother. 57:2506-2510.

Synergy is observed where there is a reduction in MIC compared to chemotherapeutic drug or protein biologic alone.

FIC value is calculated (Fractional Inhibitory concentration) of two agents A and B is calculated as FIC index=FIC-A+FIC-B

FIC-A=MIC of A in combination/MIC of A alone.

FIC-B=MIC of B in combination/MIC of B alone.

As used here, a FIC index value of <0.5 indicates “synergy” in inhibitory activity, though a different ratio can be selected as sufficient in other embodiments.

Synergy was determined by CFU based assay. Using varying concentrations of Lys or anti-TB drugs individually, a concentration was selected which inhibited the growth, but did not show any bactericidal activity as seen by CFU detection in agar plates: Next, the same concentrations of LysB and anti-TB drugs were tested in combination and the cultures exposed to the combinations were plated for enumeration of CFU. Bactericidal activity (>2 log CFU reduction) was taken as an evidence of synergy between LysB and anti-TB drugs. Similar testing can be carried out in liquid cultures.

ONE: MIC of LysB on M. smegmatis and M. bovis BCG in Various Media

| | MIC (μg/ml) in | | |
|---|---|---|---|
| | various media | | |
| Organism | 7H9 | Dubos | Sauton's |
| M. smegmatis | 3 | 12 | 12 |
| M. bovis BCG | 0.40 | 0.25 | 0.50 |

TWO: Synergy of LysB with Anti-TB Drugs in M. smegmatis and M. bovis Broth Checkerboard MIC

| | M. smegmatis (7H9) | | | |
|---|---|---|---|---|
| Protein or | MIC | | M. bovis BCG (Dubos) | |
| compound | (μg/ml) | FIC index* | MIC (μg/ml) | FIC index* |
| LysB | 3 | | 0.25 | |
| INH | 8 | 0.13 | 0.03 | 0.47 |
| Rifampicin | 4 | 0.06 | 0.004 | 0.37 |
| Ethambutol | 2 | 0.06 | 16 | 0.15 |

*FIC index value of <0.5 indicates synergy

THREE: Synergy of LysB with Anti-TB Drugs in M. smegmatis by CFU Reduction Assay in 24 Hrs.

Log10 CFU reduction

| Rif | | | INH | | | Ethambutol | | |
|---|---|---|---|---|---|---|---|---|
| | | LysB + | | | LysB + | | | LysB + |
| LysB | Rif | Rif | Lysb | INH | INH | LysB | Eth | Eth |
| <1 | <1 | 5 | <1 | <1 | 3 | <1 | <1 | 5 |

FOUR: Killing of M. smegmatis by LysB Under Replicating (7H9 Broth) and Non-Replicating Conditions (Tris and Saline)

| | Log10 CFU reduction in | | |
|---|---|---|---|
| LyB concentration | 25 mM Tris | 7H9 | Saline |
| 100 μg/ml | 7 | 4 | 1 |
| 50 μg/ml | 4 | Not Done | Not done |

B. Testing for in vivo synergy

For detecting in vivo synergy, a strategy similar to outlined above using CFU reduction assay is used. Animals or subjects infected with Mycobacteria are treated with bacteriostatic concentrations of chemotherapeutics, e.g., Rif, INH or Ethambutol alone and in combination with a biologic, e.g., LysB for a selected specific duration. Demonstration of no significant reduction of CFU (e.g., <0.5 log) with compounds or LysB alone, but a significant reduction in CFU (e.g., >2 log) in combination of LysB with anti-TB drugs are evidence of synergistic action in vivo.

C. Biologic Deactivation

The LysB protein is subjected to denaturation, e.g., by heating, in a boiling water bath for, e.g., 15 minutes. The denatured and native protein is tested for enzymatic activity, MIC, bactericidal activity, permeability increasing activity, and synergy with anti-TB drugs. Integrity of the polypeptide chain is evaluated, e.g., by SDS-PAGE.

The loss or reduction in activity upon denaturation in these assays evidences a conformational requirement for the activity of LysB. The SDS-PAGE or sequence analyses can confirm integrity of the biologic molecule.

### V. Mycobacteria Outer Membrane Barrier Assay

A. Polypeptide Release Assay; from periplasmic space to extracellular solution

Nitrocefin based outer membrane permeabilization assay: The nitrocefin based assay measures release of β-lactamase from the periplasmic space into exterior of bacteria. Flores, et al. (2005) “Genetic analysis of the β-lactamases of Mycobacterium tuberculosis and Mycobacterium smegmatis and susceptibility to β-lactam Antibiotics” Microbiology 151:521-532. While the β-lactamase is a protein of significant molecular weight, this is a relatively insensitive assay for outer membrane permeability. Sufficient permeability for the large polypeptide to be released is indicative of a dramatic effect on outer membrane integrity. Careful controls were performed to minimize effects of leakage, amount of test enzyme, time of treatment, cell density, buffers, etc. Thus, various candidate entities may be screened for ability to compromise mycobacteria outer membrane integrity, and successful candidates may be characterized and optimized to provide efficient permeability increase for small molecule antibiotics. Screening genomic libraries coding for proteins with the desired properties or expression of individual gene products predicted to have permeability effects on the outer membrane, e.g., lipase or esterase, or suggested candidate groups described above. The screening methods can either detect the enzymatic activity or the phenotypic effect on the mycobacterial cell (e.g., outer membrane permeability effects such as release of markers such as cytoplasmic ATP or periplasmic β-lactamase).

LysB mediated increase in permeability, which was measured by exposing M. smegmatis cells to LysB in appropriate amounts for appropriate time periods under controlled conditions. Detecting β-lactamase activity in the culture supernatant measured amount of release, which was compared to absence of outer membrane acting biologic. Untreated cell controls were included in the assay to check the basal level of β-lactamase release in the culture supernatant.

Assay conditions used: 100 μl of 0.6 OD600 M. smegmatis culture (˜10E7 CFU/ml) were washed twice with Tris buffer (25 mM, pH 7.5) and incubated with LysB (25 μg/ml) for 4 hrs at 37° C. After 4 hrs of incubation, samples were centrifuged at 13K for 5 min at RT and supernatant was taken for assay. The assay mixture consisting of 100 μl of sup+100 μl of nitrocefin (250 μg/ml) was incubated for 30 min at RT in dark for color development. OD was measured at 480 nm. The following ODs were obtained in the assay. See Flores, et al. (2005) Microbiology 151:521-532.

Untreated=0.261; LysB treated=0.832

The data indicated that the LysB-treated cells leaked significantly more β-lactamase than untreated cells. Synergy studies were then carried out.

Similar assays may be applied with higher throughput screening of candidate permeabilizing activities, or specific candidates. The assays may be modified or optimized for sensitivity, as appropriate.

B. Other Release Assays

Smaller polypeptide reporters could also be substituted for the β-lactamaseenzyme. Release amounts could be evaluated by, e.g., immunological methods, including ELISA, or other enzymatic assays if the peptide is similarly an enzyme. A more sensitive assay for enzyme release may depend upon activity, supplying an enzyme substrate for the reporter. A colorimetric assay may be used for screening for release of marker, at greater sensitivity, e.g., in which a lipase or esterase will act on para-nitrophenol butyrate (PNPB) as substrate to release PNP.

Alternatively, a lower molecular weight reporter can be used. A dye or smaller molecule can be introduced into the periplasmic space, e.g., fluorescein. Free reporter is washed away so there is no release when the outer membrane remains intact. Treatment of the outer membrane with a biologic which causes release of the dye is an assay for action on the outer membrane. The speed and properties of the permeability effects can be compared for different biologics and using different reporters or dyes.

Direct detection of peptidoglycan degradation products can indicate permeability effects on the outer membrane. For example, assays for release of using the specific substrate (mycolyl-arabinogalactan—peptidoglycan (mAGP) isolated from mycobacterial cell walls may detect accessibility of peptidoglycan acting enzymes to their substrates, indicating disruption of outer membrane integrity.

C. Reporter Uptake Assays

Other assays detecting increase in permeability include uptake of ethidium bromide, malachite green, FITC, 14C precursors and assays based on a dye color change upon entering into a periplasmic space pH or oxidation state gradient. These assays detect increased entry of fluorescent or radioactive marker past the outer membrane barrier, e.g., into the periplasmic space or into the cell upon increased permeability of the mycobacteria outer membrane. See, e.g., Banaei, et al. (2009) “Lipoprotein Processing Is Essential for Resistance of Mycobacterium tuberculosis to Malachite Green” Antimicrobial Agents and Chemotherapy 53:3799-3802; Laneelle and Daffe (2009) “Transport assays and permeability in pathogenic mycobacteria” Methods Mol. Biol. 465:143-51; Rodrigues, et al. (2011) “Ethidium bromide transport across Mycobacterium smegmatis cell-wall: correlation with antibiotic resistance” BMC Microbiology 11:35; and Backus, et al (2011) “Uptake of unnatural trehalose analogs as a reporter for Mycobacterium tuberculosis” Nat. Chem. Biol. 7:228-235. Dyes whose color changes with pH or oxidation state differences near the cell compared to far away can be more sensitive than some other quantitation methods. The cells can be dead, dormant, or active and the assay modified accordingly.

Other reporters which can be used include metabolites or signaling molecules which signal the cell to respond. Here, active cells can respond to the reporter reaching the cell, and the cell response can be monitored.

D. Reporter Release Assays

In other embodiments, the membrane perturbing action may cause release of other reporters from the periplasmic space through the mycobacteria outer membrane. The reporter may be loaded into the cell by appropriate methods, or produced by the cell by appropriate culturing methods or the like. Alternatively, the membrane perturbing activity may cause lysis and release of cellular contents into the extracellular solution, which may be evaluated by sensitive detection methods. The cellular contents may be appropriately labeled or otherwise selected to be detectable when cell lysis is effected.

### VI. Assays for Screening for Activities on Macrophages or Granuloma Barriers

A. Screening for Biologics Increasing Accessibility to Lysosomes within Macrophages; Across Fibroblast Barriers

For facilitating entry of LysB or chemotherapeutics into intracellular compartments of eukaryotic cells where mycobacteria could be residing (macrophages, fibroblast, other cells), the cell penetrating peptides, CPPs, from mycobacterial or non-mycobacterial sources will be fused or conjugated, either covalently or non-covalently. to another reporter, e.g., LysB, which can be at the N-terminus or C-terminus. See Lu, et al. (2006) “A cell-penetrating peptide derived from mammalian cell uptake protein of Mycobacterium tuberculosis” Analytical Biochemistry 353:7-14; El-Shazly, et al. (2007) “Internalization by HeLa cells of latex beads coated with mammalian cell entry (Mce) proteins encoded by the mce3 operon of Mycobacterium tuberculosis” Journal of Medical Microbiology 56:1145-1151; and Heitz, et al. (2009) “Twenty years of cell-penetrating peptides: from molecular mechanisms to therapeutics” British Journal of Pharmacology 157:195-206.

Entry of a fusion protein into the intracellular compartment can be detected by known methods such as fluorescence based methods in various eukaryotic cell lines. The examples of cell lines include HeLa cells, MDCK, macrophage cell line, human fibroblast, etc. For checking cellular uptake of the protein, the cells will be treated with a known concentration of Fluorescein-labeled fusion protein and the extracellular protein will be washed off. The uptake of the protein by the cells will be monitored by fluorescence microscopy.

B. Bead Assays

Eukaryotic cell permeability assays can be developed using latex beads coated with a protein. An aliquot of the latex beads coated with various fusion proteins (e.g. Mce3 fusion) is added to eukaryotic cell (e.g., HeLa cell) monolayer, e.g., in a six-well plate. The cells are incubated for varying time periods (e.g., 15 min to 4 hrs) at 37° C. The cells are washed with cell culture medium (MEM), fixed with 100% methanol and stained with 1% Evans Blue for observing under light and phase-contrast fluorescence microscopy. Alternatively, fluorescence emissions (excitation at 590 nm and emission at 630 nm) are detected in the 96-well plate using a fluorometer to evaluate how many beads have been internalized. El-Shazly, et al. (2007) “Internalization by HeLa cells of latex beads coated with mammalian cell entry (Mce) proteins encoded by the mce3 operon of Mycobacterium tuberculosis” Journal of Medical Microbiology 56:1145-1151.

C. Accumulation Assays

Certain compounds (e.g., ciprofloxacin) are known to be ineffective in intracellular assays because of permeability or efflux issues. Shandil, et al. (2007) “Moxifloxacin, Ofloxacin, Sparfloxacin, and Ciprofloxacin against Mycobacterium tuberculosis: Evaluation of In Vitro and Pharmacodynamic Indices That Best Predict In Vivo Efficacy” Antimicrobial Agents and Chemotherapy, February 2007, p. 576-582. Biologics which can increase the permeability of eukaryotic cells can potentiate the bactericidal effect of Ciprofloxacin, and likely can similarly mediate the effect of other mycobacterial chemotherapeutics.

As described above, various types of efflux disrupters may be candidates for also disrupting the integrity of the granuloma barriers protecting the mycobacteria within the macrophages and/or granulomas. Screening methods can be applied, as in the mycobacteria outer membrane disruption screening, for genes or variants thereof which will provide accessibility to the contents of the granulomas for the therapeutic compositions and combinations of the invention. The accessibility of the compositions into the granulomas, and intracellularly into the macrophages, to reach the mycobacteria within will increase susceptibility of the mycobacteria to the combination compositions described herein.

1. A method for treating a subject having a mycobacterial infection, comprising administering to the subject:
a) an outer membrane acting biologic; and
b) a mycobacterial chemotherapeutic,
wherein the outer membrane acting biologic and mycobacterial therapeutic act synergistically to reduce mycobacteria, thereby treating the subject.

2. The method of claim 1, wherein the outer membrane acting biologic is not LysA, or a fragment thereof having LysA activity.

3. The method of claim 1, wherein less than 90% of the standard dose of mycobacterial chemotherapeutic is administered or the mycobacterial chemotherapeutic is administered for less than 90% of the standard duration of treatment.

4. The method of claim 1, wherein the Fractional Inhibitory Concentration (FIC) index is less than 0.6.

5. The method of any one of the foregoing claims, wherein the outer membrane acting biologic and mycobacterial chemotherapeutic are both administered within a period of 2 days.

6. The method of claim 1, wherein the outer membrane acting biologic is administered before the mycobacterial chemotherapeutic or both are administered simultaneously.

7. The method of claim 1, wherein the outer membrane acting biologic, mycobacterial chemotherapeutic, or both are administered topically or in a slow release formulation.

8. The method of claim 1, further comprising administering
a) a biologic or therapeutic compound which increases accessibility of the mycobacterial chemotherapeutic to a mycobacterial cell inside a macrophage or monocyte;
b) a biologic or therapeutic compound which increases accessibility of the mycobacterial chemotherapeutic to the interior of a granuloma; or
c) a biologic or therapeutic compound that increases permeability of a macrophage or monocyte.

9. The method of claim 1, wherein the subject:
a) is immunosuppressed;
b) is HIV positive;
c) has been diagnosed with cystic fibrosis, alpha-1 antitrypsin deficiency, Marfan's syndrome, or Primary Ciliary Dyskenesia; or
d) is a mammal, reptile, amphibian, or fish.

10. The method of claim 1, wherein the mycobacterial infection is:
a) a tuberculosis infection;
b) environmental mycobacteria;
c) atypical mycobacteria;
d) a mycobacteria other than tuberculosis (MOTT) or non-tuberculosis mycobacteria (NTMB);
e) latent;
f) active;
g) disseminated;
h) extrapulmonary or lymphatic;
i) multidrug resistant; or
j) a post-traumatic abscess, swimming pool granuloma, Buruli ulcer, or leprosy.

11. The method of claim 1, wherein the outer membrane acting biologic:
a) decreases the amount of mycobacterial chemotherapeutic or duration of mycobacterial chemotherapeutic treatment required to treat the mycobacterial infection in the subject;
b) increases permeability of a granuloma;
c) acts on a mycolic acid or lipoarabinomanin;
d) acts on a mycobacterial cell wall or outer membrane;
e) is an esterase;
f) is a lipase;
g) is a cutinase;
i) is an alpha/beta hydrolase;
j) is mycolylarabinogalactan esterase;
k) is phage LysB;
l) is D29 phage LysB; or
m) is in a sterile or buffered formulation.

12. The method of claim 1, further comprising administering a therapeutic compound that increases the permeability of a macrophage, monocyte, or granuloma.

13. The method of claim 1, wherein said mycobacterial chemotherapeutic is selected from the group consisting of:
a) a front line mycobacterial therapeutic such as isoniazid, pyrazinamide, ethambutol, or rifampin;
b) a second line mycobacterial therapeutic such as a fluoroquinolone (e.g., ciprofloxacin, levefloxacin, moxifloxacin), a cyclic peptide (e.g., capromycin, viomycin, enviomycin), a thioamide (e.g., ethionamide, prothionamide), cycloserine, terizidone, an aminoglycoside, PAS, kanamycin, capreomycin, amikacin, or streptomycin;
c) a third or subsequent line mycobacterial therapeutic;
d) one of a microlide, a β-lactam, a β-lactamase inhibitor, clavulenic acid, trimethoprim, or sulfamethoxazole;
e) one of clarithromycin, rifampicin, rifabutin, amikacin, azithromycin, or moxifloxacin; and
f) one of diarylquinoline, dedaquiline, TMC207, a nitroimdazole, PA-824, OPC-67683, an oxazolidinone, linezolid, sutezolid, AZD5847, BTZ043, and SQ109.

14. The method of claim 1, wherein the administering results in
a) reduced mycobacterial levels in the subject with a lower dose of mycobacterial chemotherapeutic than would be required in the absence of the outer membrane acting biologic;
b) reduced mycobacterial levels in the subject with a shorter duration of treatment with the mycobacterial chemotherapeutic than would be required in the absence of the outer membrane acting biologic;
c) reduced side effects;
d) reduced mycobacterial levels in the subject with a reduced number of mycobacterial chemotherapeutics than would be required in the absence of the outer membrane acting biologic.

15. A kit, compartment, or therapeutic composition comprising:
a) an outer membrane acting biologic; and
b) a mycobacterial chemotherapeutic.

16. The kit, compartment, or therapeutic composition of claim 15, wherein the outer membrane acting biologic and mycobacterial therapeutic act synergistically to reduce mycobacteria.

17. The kit, compartment, or therapeutic composition of claim 15, wherein the kit comprises a compartment holding outer membrane acting biologic and a compartment holding the mycobacterial chemotherapeutic.

18. The kit, compartment, or therapeutic composition of claim 15, wherein the kit, compartment, or therapeutic composition is packaged in single dosage form.

19. A method for treating a subject having a mycobacterial infection, comprising administering to the subject a LysB biologic, wherein the LysB biologic reduces mycobacteria, thereby treating the subject.

20. The method of claim 19, further comprising administering a mycobacterial chemotherapeutic.

21. The method of claim 19, wherein the mycobacterial infection is tuberculosis.
